(19) United States
(12) Patent Application Publication
(10) Pub. No.: US 2026/0166185 A1
(43) Pub. Date: Jun. 18, 2026

(54) BISPECIFIC ANTIBODY COMPOUNDS

(71) Applicant: Y-mAbs Therapeutics, Inc., Princeton, NJ (US)

(72) Inventors: Søren CHRISTENSEN, Jyllinge (DK); Nico LIEBENBERG, Fredensborg (DK); Stine Louise Reedtz KJELLEV, Birkerød (DK); Steen LISBY, Hørsholm (DK); Finn Christoph WIBERG, Farum (DK); Mette GILLBERG, Hørsholm (DK); Torben LUND-HANSEN, Roskilde (DK)

(21) Appl. No.: 19/124,011

(22) PCT Filed: Nov. 7, 2023
(86) PCT No.: PCT/DK2023/050269

§ 371 Date: Apr. 24, 2025

(30) Foreign Application Priority Data
Nov. 8, 2022 (DK) PA 2022 70540

(51) Int. Cl. A61K 51/10; A61K 39/00; C07K 16/28; C07K 16/32; C07K 16/44

(52) U.S. Cl. CPC A61K 51/109; A61K 51/1093; C07K 16/2896; C07K 16/32; C07K 16/44; A61K 2039/505; A61K 2121/00; A61K 2123/00; C07K 2317/14; C07K 2317/31; C07K 2317/55; C07K 2317/569; C07K 2317/92

(57) ABSTRACT

Disclosed are new compounds comprising two antigen binding sites and a tetramerization site, where the antigen binding sites are selected among natural binding sites. The new compounds have improved manufacturability, stability and solubility compared with similar compounds based on scFv binding sites, making the disclosed compound much better candidates for active ingredients in pharmaceutical compositions.

The present specification comprises a sequence listing in computer readable format, submitted together with the application. The sequence listing forms part of the disclosure and is incorporated in the specification in its entirety.

The present invention relates to bispecific antibodies for use in radioimmunotherapy, in particular bispecific antibodies capable of di- or tetramerizing, and comprising two antigen binding sites, one capable of binding a tumor antigen and one capable of binding a chelator molecule with or without a radionuclide.

### TECHNICAL BACKGROUND

Pretargeted radioimmunotherapy (PRIT) is a useful method for treating solid cancers using a bispecific antibody having an antigen binding site capable of binding an antigen exposed on the surface of the tumor and further an antigen binding site capable of binding a radionuclide or a chelator wherein a radionuclide is bound.

PRIT is performed by first administering the bispecific antibody to the patient, and when the bispecific antibody has bound to the tumor, a chelator with a bound radionuclide is administered and will be bound by the bispecific antibody and thereby be localized to the tumor. It is beneficial to delay administering the chelator with the radionuclide until the bispecific antibody has been cleared from circulation in order to reduce systemic exposure to radiation. Some protocols use an additional step administering a clearing agent between administering the bispecific antibody and the radionuclide, in order to facilitate the clearing of the bispecific antibody.

Many different formats of the bispecific antibody have been used for PRIT, of which some rely on the use of single chain variable fragments (scFv).

WO 2018/204873 discloses bispecific antibodies for PRIT comprising a scFv capable of binding a tumor antigen, a scFv capable of binding DOTA chelated to a metal ion and a tetramerization domain. These bispecific antibodies have the ability to exist in a monomeric form and a multimeric form depending on concentration, meaning that they can be administered in multimeric form and when the administered antibodies are diluted in the blood stream of the patient, they will be converted into the monomeric form, which facilitate clearing from the plasma via the kidneys. Therefore, the bispecific antibodies disclosed can be used without the need of administering a clearing agent.

ScFvs are synthetic binding sites comprising the variable fragment of the light and heavy chains of an antibody, and the format has found wide used in many applications. However, experience has shown that there are a number of challenges connected to the scFv format, such as low production yield, low stability and low solubility.

These challenges have in the past been partly solved using protein engineering techniques such as introducing a stabilizing disulfide bond between the light variable chain and the heavy variable chain of an scFv, introducing one or more additional substitutions or engineering the host cell producing the scFv (see Kang and Seong in Frontiers in Microbiology (2020) vol 11, Article 1927).

Despite recent developments there is still a need for bispecific antibodies for PRIT having improved manufacturability compared to previously designed bispecific antibodies.

### SUMMARY OF THE INVENTION

The invention relates to a compound comprising a first antigen binding site capable of binding a tumor antigen, a second antigen binding site capable of binding a chelator wherein one of the first and/or the second antigen binding sites is a VHH fragment and the other of the first and/or the second antigen binding site is selected among Fab fragments and VHH binding fragments.

In some embodiments the compound further comprises a tetramerization domain and/or a third or subsequent antigen binding site.

In another aspect the invention relates to a composition comprising the compound of the invention.

In another aspect the invention relates to a method of treating and/or diagnosing cancer comprising the steps of
a. Administering a compound according to the invention or a composition according to the invention to a person in need of treatment and/or diagnosis.
b. Administering a radionuclide bound to a chelator that can be bound by the second antigen binding site.

In case that said method is a method for diagnosing cancer, the method may further comprise a step of detecting the radioactivity, such as using a scanning step.

The invention also relates to a nucleic acid encoding a compound of the invention, an expression vector comprising the nucleic acid of the invention, a host cell comprising the nucleic acid of the invention and a method of producing the compound of the invention.

### DETAILED DISCLOSURE

### Definitions

Amino acid alteration: The term “amino acid alteration” is intended to mean an alteration of one amino acid found in the original amino acid sequence, where the alteration is selected among a substitution, a deletion or an insertion of an additional amino acid immediately after the amino acid in question.
Amino acid substitution: The term “amino acid substitution” is intended to mean the replacement of one amino acid with a different amino acid. In the present specification the term amino acid substitution(s) with reference to a reference sequence is intended to mean that the amino acid sequence in question can be generated starting from the reference sequence and introducing said amino acid substitution(s), even if the sequence in question actually was generated by another process not involving the reference sequence.
Antibody: The term “antibody” is art-recognized terminology and is intended to include molecules or active fragments of molecules that bind to an antigen. Natural antibodies, except heavy chain antibodies, are composed of two heavy chains, each comprising one variable domain and three or more constant domains (CH1, CH2 and CH3), and two light chains, each comprising one variable domain and one constant domain (CL). One light chain is bound to one heavy chain by a disulfide bond localized in the constant domains (CH1 and CL), and the two heavy chains are bound to each other by a number of disulfide bonds localized in the constant domains (CH2).

Several isotypes of natural antibodies are known including IgA, IgD, IgE, IgG such as IgG1, IgG2, IgG3, and IgM, and these isotypes mainly differ in the constant domains.
Fab fragment: The term “Fab” or “Fab fragment” is an antibody fragment consisting of a first polypeptide comprising the light chain variable domain and the light chain constant domain and a second polypeptide comprising the heavy chain variable domain and the heavy chain constant domain, where the two polypeptides are connected via a disulfide bond localized in the constant regions. A Fab fragment can be provided by proteolytic cleavage of a natural antibody, or it can be made by expressing and combining the two polypeptides, e.g. using recombinant DNA technologies as known in the area. A Fab fragment is capable of binding to the same antigen that is recognized by the intact antibody. The term “Fab” or “Fab fragment” encompass both natural Fab fragments, i.e. fragments having same sequence as found in an natural antibody; and synthetic or engineered Fab fragments; i.e. fragments having same overall structure as a natural Fab Fragment, but where the sequence has been engineered by introducing one or more amino acid alterations, such as substitutions, deletions or insertions; in one or both amino acid chains.
Bispecific antibody: A bispecific antibody is an antibody that can bind simultaneously to two targets which are of different structure. Bispecific antibodies (BsAb) are non-natural, engineered antibodies that have at least one binding site that specifically binds to one antigen, for example a tumor antigen, and at least one other binding site that specifically binds to another antigen, for example a chelator binding a radionuclide. A variety of bispecific fusion proteins can be produced using molecular engineering. In one form, the bispecific fusion protein is divalent, consisting of, for example, a VHH with a single binding site for one antigen and a Fab fragment with a single binding site for a second antigen. In another form, the bispecific fusion protein is tetravalent, consisting of, for example, an IgG with two binding sites for one antigen and two identical scFvs for a second antigen.
CDR: Complementarity Determining Regions (CDR) are part of the variable regions of antibodies and are of key importance for the binding specificity of an antibody. A typical antibody consisting of two heavy chains and two light chains has 6 CDR sequences, three in the light chain and three in the heavy chain.
DOTA: DOTA (Dodecane Tetraacetic Acid) is also referred to as 1,4,7,10-tetraazacyclododecane-1,4,7 10-tetraacetic acid, and has the formula (CH₂CH₂NCH₂CO₂H)₄ also known as C₁₆H₂₈N₄O₈·xH₂O.
Derivative of DOTA: is intended to mean a compound comprising the DOTA ring system with some other chemical group or moiety attached and is capable of chelating metal ions. Examples of such compounds include Benzyl-DOTA and the bispecific chelators disclosed in WO2019010299A (Proteus-DOTA) or WO2022005998. Additional DOTA derivatives are disclosed in WO2010099536 A1.
DOTAM: is a chelator comprising a ring system capable of binding metal ions. It has the systematic name of 1,4,7,10-Tetraazacyclododecane-1,7-bis(acetate)-4,10-bis(acetamide) and has the formula C₁₆H₃₀N₆O₆.2H₂O
Effective amount: As used herein, the term “effective amount” refers to an amount of a given compound or composition that is necessary or sufficient to realize a desired biologic effect. An effective amount of a given compound or composition in accordance with the methods of the present invention would be the amount that achieves this selected result, and such an amount can be determined as a matter of routine by a person skilled in the art, without the need for undue experimentation.
Plasma half-life: The term “plasma half-life” for a given compound is the time required for plasma concentration of a given compound to be diminished by 50%. The plasma half-life depends on different factors and properties of the given compound. An important factor for the plasma half-life is the size, as it is known that the kidneys have a filtering function that retains molecules having a size of about 70 kDa, whereas smaller molecules may be eliminated via the kidneys. Further, some molecules may interact with receptors which may affect plasma half-life.
Prevent: As used herein, the terms “prevent”, “preventing” and “prevention” refer to the prevention of the recurrence or onset of one or more symptoms of a disorder in a subject as result of the administration of a prophylactic or therapeutic agent.

Radioactive isotope: Examples of radioactive isotopes that can be bound to antibodies. e.g. by conjugation or the use of a chelator; for use diagnostically or therapeutically include, but are not limited to, ²¹¹At, ¹⁴C, ⁵¹Cr, ⁵⁷Co, ⁵⁸Co, ⁶⁷Cu, ¹⁶⁵Dy ¹⁵²Eu, ⁶⁷Ga, ³H, ¹¹¹In, ⁵⁹Fe, ¹³³La, 177Lu, ³²P, ²²³Ra, ²²⁴Ra, ¹⁸⁶Re, ¹⁸⁸Re, ⁷⁵Se, ⁸⁹Sr¹⁴⁹Tb, ¹⁵¹Tb, ¹⁶¹Tb^{99m}Tc, ²²⁷Th, ⁸⁹Zr, ⁹⁰Y, ¹²³I, ¹²⁴I, ¹²⁵I, ¹³¹I, ^{94m}Tc, ⁶⁴Cu, ⁶⁸Ga, ⁶⁶Ga, ⁷⁶Br, ⁸⁶Y, ⁸²Rb, ^{110m}In, ¹³N, ¹¹C, ¹⁸F and alpha-emitting particles. Non-limiting examples of alpha-emitting particles include ²⁰⁹Bi, ²¹¹Bi, ²¹²Bi, ²¹³Bi, ²¹²Pb, ²¹⁰Po, ²¹¹Po, ²¹²Po, ²¹⁴Po, ²¹⁵Po, ²¹⁶Po, ²¹⁸Po, ²¹¹At, ²¹⁵At, ²¹⁷At, ²¹⁸At, ²²¹Fr, ²²³Ra, ²²⁴Ra, ²²⁶Ra, ²²⁵Ac, ²²⁷Ac, ²²⁷Th, ²²⁸Th, ²²⁹Th, ²³⁰Th, ²³²Th, ²³¹Pa, ²³⁷Np, ²³⁸Pu, ²³⁹Pu, ²⁴⁰Pu, ²⁴⁴Pu, ²⁴¹Am, ²⁴⁴Cm, ²⁴⁵Cm, ²⁴⁸Cm, ²⁴⁹Cf, and ²⁵²Cf.
Sequence Alignment: Sequence alignment refers simply to any way to align two sequences one below another. It is a way of arranging sequences of DNA, RNA or protein to identify regions of similarity between the sequences. Different alignment algorithms exist and they usually have a scoring function which assigns every alignment a numeric score indicating how good an alignment is and tries to find the best alignment according to its scoring function.
Sequence identity: The term Sequence identity is intended to mean a measurement of the relatedness of two nucleic or amino acid sequences. Sequence identity is determined by aligning the two sequences and finding the longest overlap, counting the number of matches in the overlap and calculating the sequence identity by dividing the number of matches by the number of, nucleotide or amino acid, residues in the overlap. Sequence identity is typically expressed in percent (%).

A variety of computational algorithms are available for the skilled person, for generating sequence alignment and calculating Sequence identity. As used herein, Sequence alignment refers to Pairwise alignments. Several algorithms perform this including the sequence alignment program Clustal Omega [doi: 10.1038/msb.2011.75].

As used herein the sequence alignment may refer to the following algorithm and parameters:
Algorithm: Clustal Omega (1.2.4), http://www.clustal.org/omega/

Heavy chain antibody is an antibody comprising two heavy chains and lacks the two light chains usually found in natural antibodies. Heavy chain antibodies are naturally found in members of the camelid order and further in cartilaginous fish such as some sharks.

Structurally, heavy chain antibodies are composed of a single variable domain comprising the complementary determining regions (CDR), and 2 to 5 constant domains, depending on their origin.

Heavy chain antibodies derived from camelids comprises two constant regions, whereas heavy chain antibodies derived from cartilaginous fish may have up to five constant domains.

Single domain antibody (sdAb) or VHH fragment is a fragment of a heavy chain antibody, comprising the variable domain, i.e., the antigen binding domain; of a heavy chain antibody but lacks the constant domains. A VHH fragment, also called a nanobody, is a rather small molecule with a molecular weight below 20 kDa. The terms Single domain antibody (sdAb), VHH fragment, VHH, VHH binder or nanobody may be used interchangeably throughout this description and claims.

Treatment: As used herein, the terms “treatment”, “treat”, “treated” or “treating” refer to prophylaxis and/or therapy, particularly wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

Pharmaceutical composition: As used herein the term “Pharmaceutical composition” is intended to mean a composition intended for administration as a drug or medicine to a patient in need thereof. Pharmaceutical compositions comprise at least one active ingredient and at least one pharmaceutical grade ingredient such as solvents, diluents, salts, stabilizing agents, pH regulating agents, antioxidants etc. Pharmaceutical compositions are prepared from pharmaceutical grade ingredients e.g., as described in European Pharmacopoeia 10^{th} Edition, using methods and technologies known in the pharmaceutical or apothecary area.

### The Compound

The invention relates to a compound comprising a first antigen binding site and a second antigen binding site, wherein at least one of the first and the second antigen binding sites is a VHH fragment, and the other of the first and the second binding site is a VHH fragment or a Fab fragment. One of the first and the second binding site is capable of binding a tumor antigen, and the other of the first and the second binding site is capable of binding a chelator or a chelator binding a metal ion.

In some embodiments the compound of the invention further comprises a tetramerization domain.

In some embodiments the compound of the invention may even comprise a third or subsequent binding site in form of a second or subsequent VHH fragment.

The compound of the invention may have a structure, where the first binding site is a Fab fragment and the second binding site is a VHH fragment, the first binding site is a VHH fragment, and the second binding fragment is a Fab or the first binding fragment is a VHH fragment, and the second binding fragment is a VHH fragment.

In the embodiment where both the first and the second antigen binding site is in form of a VHH, the whole compound may be produced as a single polypeptide strand comprising the two binding sites and either with or without the tetramerization domain.

In the embodiment where one of the antigen binding sites is in form of a Fab and the other binding site is in form of a VHH, the compound is generally composed of two polypeptide strands and several combinations are possible.

In this embodiment the VHH fragment may be comprised in the same polypeptide strand as the light chain of the Fab or in the same polypeptide chain as the heavy chain of the Fab or on both the light chain and the heavy chain. It is even encompassed in the present invention that one VHH fragment is comprised in the same strand as the light chain of the Fab and a second VHH fragment, that may be the same or different from the first VHH fragment, is comprised in the same strand as the heavy chain of the Fab. In the latter situation the compound may comprise two binding specificities, one binding specificity provided through the Fab and one binding specificity provided by two identical VHH fragments (such a structure is herein called a homodimeric molecule) or three different binding specificities, one binding specificity provided through the Fab and two binding specificities provided by the two different VHH fragments (such a structure is herein called a heterodimeric molecule).

It is even contemplated to prepare a molecule of the invention, wherein one or both of the polypeptides of the molecule comprises one VHH fragment located in the N-terminal part of the polypeptide and a second VHH fragment located in the C-terminal part of the polypeptide. In this way it is possible to prepare molecules of the invention comprising two, three or even more specificities.

Similarly, if present, the tetramerization domain may be located in the same polypeptide strand as the light chain of the Fab or in the same polypeptide chain as the heavy chain of the Fab.

This leads to several possible combinations as the compound of this embodiment may comprise:
one polypeptide comprising a VHH domain, a light chain of the Fab and a tetramerization domain; and a second polypeptide chain comprising a heavy chain of the Fab;
one polypeptide comprising a VHH domain and a light chain of the Fab; and a second polypeptide chain comprising a heavy chain of the Fab and a tetramerization domain;
one polypeptide comprising a VHH domain, a heavy chain of the Fab and a tetramerization domain; and a second polypeptide chain comprising a light chain of the Fab;
one polypeptide comprising a VHH domain and a heavy chain of the Fab; and a second polypeptide chain comprising a light chain of the Fab and a tetramerization domain;
one polypeptide comprising a VHH domain and a heavy chain of the Fab; and a second polypeptide chain comprising a VHH domain, that may be the same VHH domain, or it may be a different VHH domain as present in the first polypeptide, and a light chain of the Fab;
one polypeptide comprising a VHH domain, a light chain of the Fab; and a second polypeptide chain comprising a heavy chain of the Fab;
one polypeptide comprising a VHH domain, a heavy chain of the Fab; and a second polypeptide chain comprising a light chain of the Fab; or
one polypeptide comprising a VHH domain and a heavy chain of the Fab; and a second polypeptide chain comprising a VHH domain, that may be the same VHH domain, or it may be a different VHH domain as present in the first polypeptide, and a light chain of the Fab and a tetramerization domain.
one polypeptide comprising a VHH domain, a heavy chain of the Fab and a tetramerization domain; and a second polypeptide chain comprising a VHH domain, that may be the same VHH domain, or it may be a different VHH domain as present in the first polypeptide, and a light chain of the Fab.

The skilled person will appreciate that even further combinations may be possible, in particular for compounds of the invention comprising a second or subsequent VHH fragment binding site.

The order of the domains in the polypeptide(s) is not decisive for the invention. On the contrary it is contemplated that a VHH fragment may be placed in the N-terminal part, in the C-terminal part, or in an internal part of the polypeptide where it is present. Further, a polypeptide forming one of the chains of the compound of the invention may even comprise two VHH fragments, e.g. one VHH fragment in both ends of the polypeptide with one chain of a Fab fragment between the two VHH fragments. Preferably the tetramerization domain is located at the C-terminal end of the polypeptide comprising this domain.

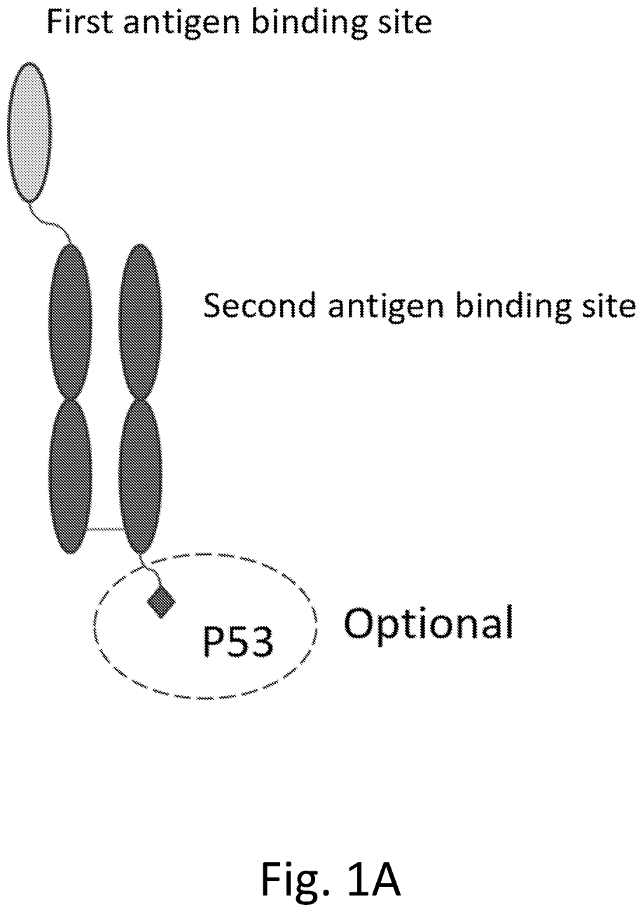

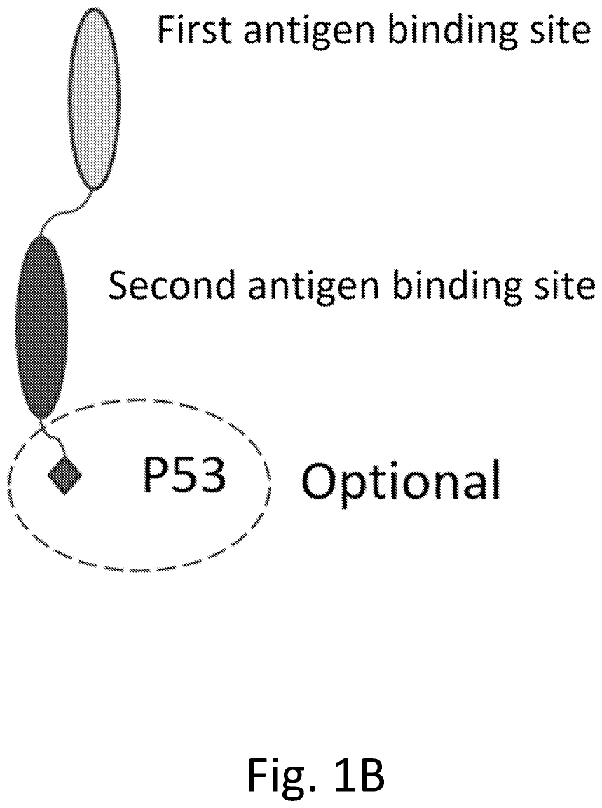

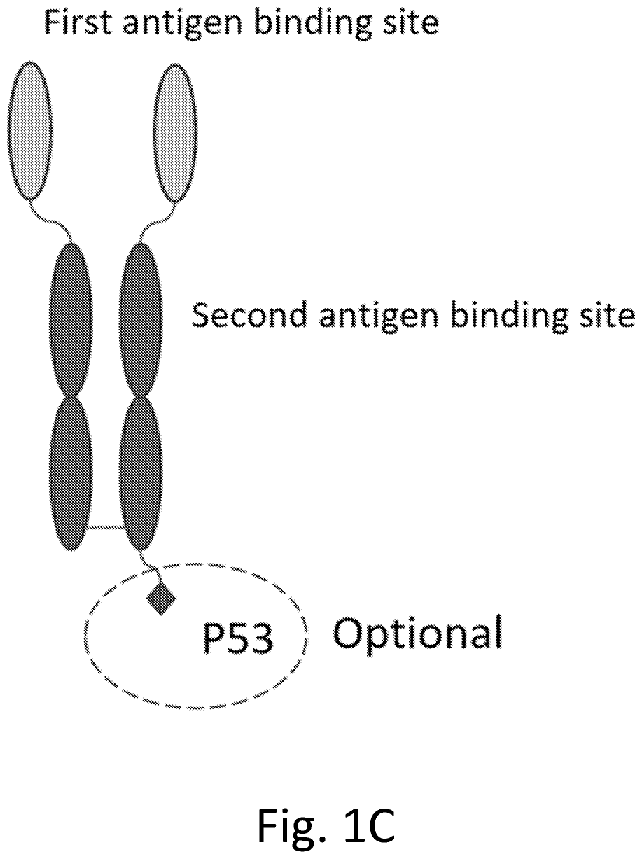

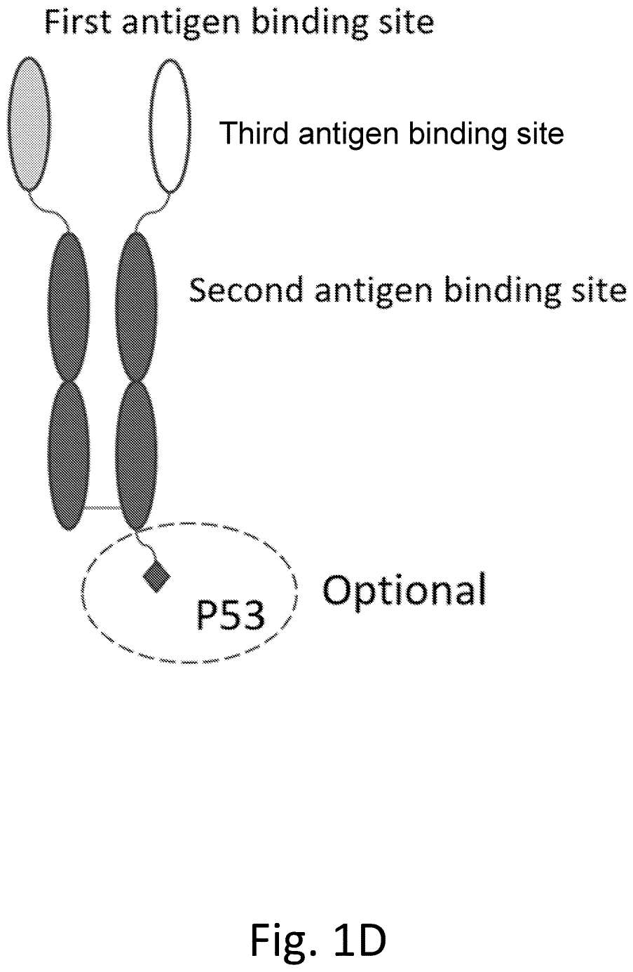

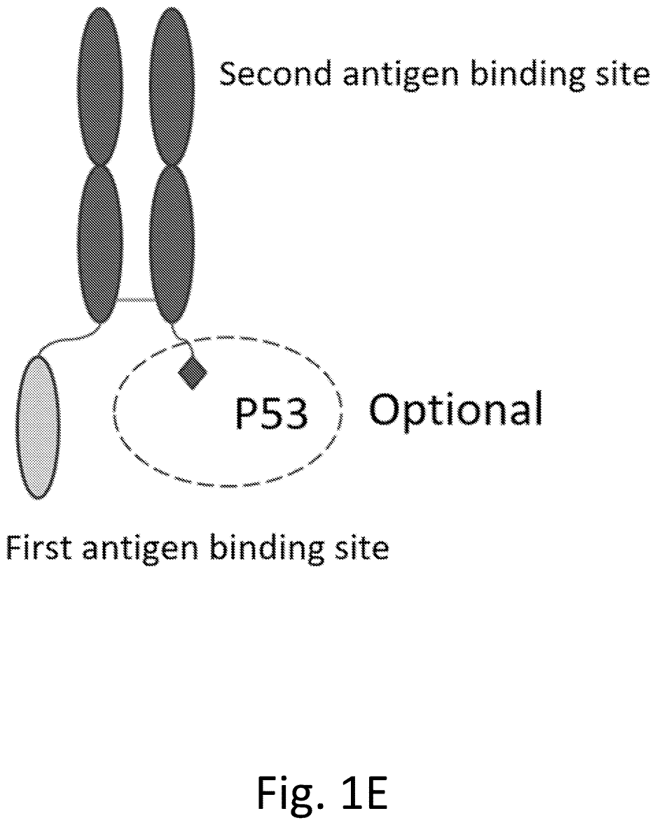

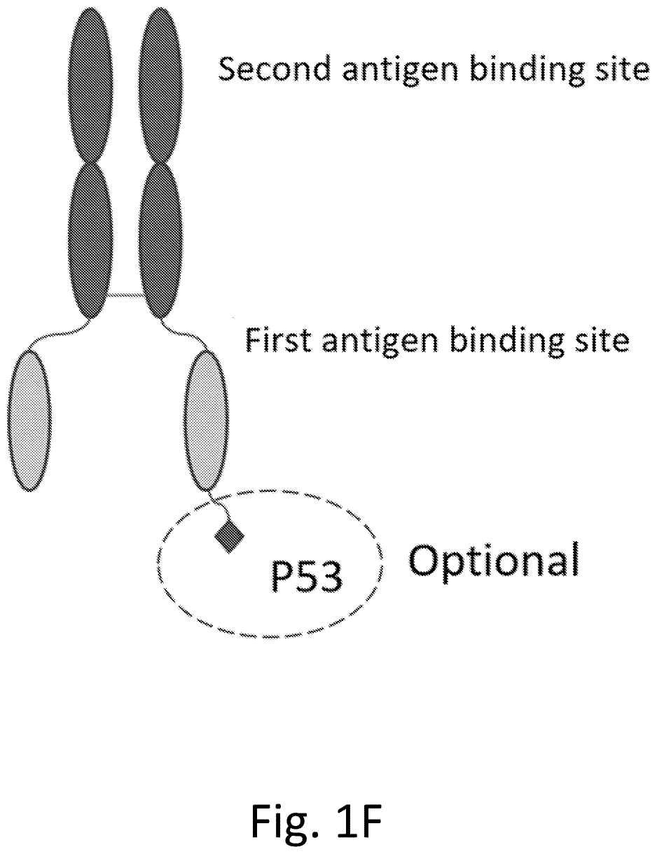

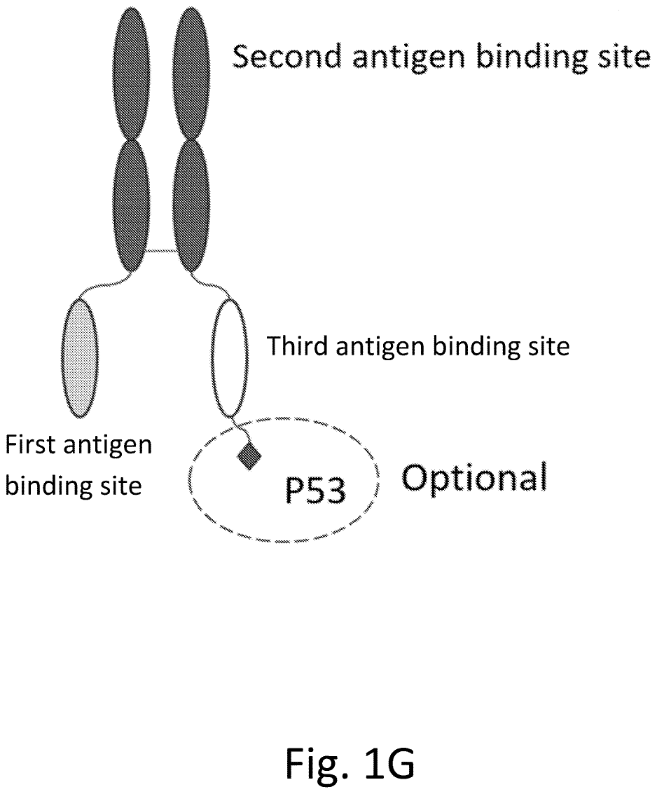

One preferred embodiment of the invention is a format where the first and the second antigen binding sites are in form of VHH fragments, and the tetramerization domain is located in the C-terminal end of the polypeptide. Such a compound is schematically shown in FIG. 1B.

Another preferred embodiment of the invention is a format where one of the first and the second antigen binding sites is a VHH and the other antigen binding site is in form of a Fab fragment wherein the tetramerization domain is localized in the C-terminal end of the first or the second polypeptide. Examples of compounds according to this embodiment are schematically shown in FIGS. 1A and 1E.

Another preferred embodiment of the invention is a format where one of the first and the second antigen biding sites is a VHH and the other antigen binding site is in form of a Fab fragment wherein the tetramerization domain is localized in the C-terminal end of the first or the second polypeptide, wherein two VHH fragments are present. The two VHH fragments may be identical, such as shown in FIGS. 1C and 1F, and in this case the avidity of the binding of the VHH fragments is increased due to the coordination of the two VHH fragment binding sites. Alternatively, the two VHH fragments may be different and capable of binding different tumor antigens, such as two different tumor antigens both expressed on the surface of the same tumor, as shown in FIGS. 1D, and 1G.

In one preferred embodiment the total molecular weight of the compound is below the renal clearance limit, such as below 70 kDa. However, the compound on tetrameric form will have a size above the renal clearance limit meaning that when the compound of the invention is administered to a patient, the compound on tetrameric form will have a long plasma half-life and the compound on monomeric form will have a short plasma half-life. Further, the compound on tetrameric form will have a higher affinity to the tumor due to the presence of four binding sites.

The tumor antigen may in principle be any antigen exposed on the tumor in a way that makes it accessible to an antibody. Many such tumor antigens are known in the art and the present invention is not limited to any particular such tumor antigen.

Examples of tumor antigens includes, but are not limited to: HER2, B7-H3, CA6, CD138, CD20, CD19, CD22, CD27L, CD30, CD33, CD37, CD38, CD47, CD56, CD66e, CD70, CD74, CD79b, EGFR, CEA, EGFRvIII, FRα, GCC, GPNMB, Mesothelin, MUC16, NaPi2b, Nectin 4, PSMA, STEAP1, Trop-2, 5T4, AGS-16, alpha v beta6, CA19.9, CAIX, CD138, CD174, CD180, CD227, CD326, CD79a, CEACAM5, CRIPTO, DLL3, DS6, Endothelin B receptor, FAP, GD2, GPA33, Mesothelin, PMEL 17, SLC44A4, TENB2, TIM-1, CD98, Endosialin/CD248/TEM1, Fibronectin Extra-domain B, LIV-1, Mucin 1, p-cadherin, peritosin, Fyn, SLTRK6, Tenascin c, VEGFR2, BAFF, BAFFR and PRLR.

Preferred tumor antigens include HER2, B7-H3, GD2, CD20, CD38, GPA33, CEA, EGFRvIII and CD33.

The antigen binding sites capable of binding a tumor antigen or capable of binding a chelator or a chelator binding a metal ion may be provided by selecting the sequences forming a Fab site or a VHH fragment from an isolated intact antibody having the desired binding properties, and expressing the sequences in suitable production cells, using methods well known in the area. Alternatively, such antigen binding sites capable of binding a tumor antigen or capable of binding a chelator or a chelator binding a metal ion may be provided by screening Fab or VHH fragment libraries for binders having the desired binding properties. This is all within the capabilities of the person skilled in the art.

In one embodiment the first antigen binding site capable of binding a tumor antigen is a Fab, which may be derived from a tumor antigen binding antibody as known in the art. By “derived from” is meant that the Fab is made of the respective fragments of the antibody from which it is derived, and optionally modified by one or more amino acid alterations, and/or it may even be humanized using methods known in the art. Examples of antibodies from which the Fab for use in the invention may be derived, includes anti-B7H3 antibody 8H9 (Modak et al (2001) Cancer. Res. 61:4048-56), anti-GD2 antibody 3F8 (Cheung et al (1998) J. Clin. Oncol. 3052-3060), anti-CD38 antibody Daratumumab (Lee (2006) Mol. Med. 12:317-23) or AT13/5 (Ellis et al. (1995) J. Immunol 155:925-37).

In one embodiment, the second antigen binding site binding a chelator is a Fab, which may be derived from a chelator binding antibody.

One preferred example of an antibody capable of binding a chelator is an antibody capable of binding DOTA, or a derivative of DOTA, e.g., as disclosed in WO 2010/099536, incorporated by reference, or derived from one of these antigen binding sites. By “derived from” is meant that the Fab is made by the respective fragment of the antibody from which it is derived, and optionally modified by one or more amino acid alterations, and/or it may even be humanized using methods known in the art. Examples of antibodies from which the Fab for use according to the invention may be derived, include the antibody designated 2D12.5 (Corneillie et al, J. Am. Chem. Soc-125:15039-15048, 2003), optionally the corresponding antibodies comprising one or more substitutions in the CDR sequences as disclosed in WO 2010/099536.

Another preferred example of an antibody capable of binding a chelator is an antibody capable of binding DOTAM; e.g. as disclosed in WO2019201959, incorporated by reference, or derived from one of these antigen binding sites.

In some preferred embodiments the compounds of the invention are further altered by selected alterations intended to improve selected properties of the compounds, e.g., remove liabilities of the compound in order to get an improved molecule.

One of the key attributes of a therapeutic monoclonal antibody or antibody derived protein candidate is that it must have the biochemical and biophysical properties that will make it stable, soluble, and not prone to degradation or modification during manufacturing and storage. A wide variety of potential liabilities may be present in a therapeutic candidate that require correction, including the propensity to aggregate, poor solubility, propensity to fragment, deamidation, oxidation, cyclization, or other chemical modification of key residues, disulphide bond shuffling, glycation, and so forth. During antibody discovery and development, it is critical to use protein sequence analysis algorithms, antibody modelling and engineering to optimize the antibodies for stability, solubility, and other biophysical characteristics. Two significant potential outcomes if antibodies are not properly behaved are immunogenicity and lack of batch-to-batch comparability.

Post-translational modifications (PTM's) of antibodies have the potential to affect affinity, stability, potency and homogeneity of an antibody, and this will result in complicated process in downstream development. The bioactivity and production of multiple isoforms of a product can potentially be impacted. PTM's normally include deamidation, isomerization, oxidation, N-glycosylation, glycation, free thiol modification, pyro-Glutamate, O-glycosylation, C-terminal Lysine removal etc.

To identify the sequences with liabilities, standard rules applied to the primary sequence of the antibody, provide a prediction of residues to modify or avoid. Several bioinformatics software packages include algorithms to provide this information. Not all PTMs can be confidently predicted by strict sequence rules and is only revealed during a deeper characterization of the antibody. (e.g., O-glycans).

Ideally a molecule should be selected from a panel of molecules. Such selection should allow only molecules with no predicted liability to proceed. However, in some cases a favorite lead candidate does hold a few less critical liabilities. These can be mitigated by generation of variants molecules of the amino acid in question. In antibodies and antibody-derived therapeutics the most critical part of the sequence are the CDRs involved in target binding. Liabilities in these sequences are likely to impact the binding and are thus often of higher priority to modify than framework liabilities.

Many such specific substitutions intended to remove identified liabilities are known in the area, and it is within the skills of the average practitioner to identify such residues and replace such residues with suitable other residues.

The tetramerization domain may be selected among any domains having the ability to tetramerize at high concentration and disassemble into monomers at low concentration. In this context “high concentration” is intended to mean the concentration that is typically found in pharmaceutical composition such as in the range of 1-50 mg/l, and “low concentration” is intended to mean the concentration of the compound in plasma after administering a dose of the compound, such as below 50 μg/l. These properties allow administration of the compound of the invention on tetrameric form, and after administration where the compound is diluted in plasma, it will gradually dissociate into monomeric form.

Examples of such tetramerization domains include the p53, p63, p73, hnRNPC, SNAP-23, StefinB, KCNQ4 and CBFA2T1 domains, having the amino acid sequences disclosed in SEQ ID NO: 1-8, and domains having at least 80% sequence identity, at least 90% sequence identity, at least 95% sequence identity; at least 96% sequence identity, at least 97% sequence identity or at least 98% sequence identity to one of SEQ ID NO: 1-8.

A preferred tetramerization domain is the p53 domain, having the sequence of SEQ ID NO:1; and domains having at least 80% sequence identity, at least 90% sequence identity, at least 95% sequence identity; or at least 97% sequence identity to SEQ ID NO: 1.

In another embodiment the tetramerization domain is a domain comprising the sequence of SEQ ID No. 1 or a sequence that differs from this sequence by 1, 2, 3, 4, 5, 6, 7, 8, 9 or 10 alterations, selected among substitutions insertions or deletions of single amino acid residues.

Preferred tetramerization domains according to the invention are domains comprising a sequence with at least 80% sequence identity at least 90% sequence identity, at least 95% sequence identity; at least 96% sequence identity to amino acids 6-36 of SEQ ID NO: 1, and which differs from the sequence of SEQ ID NO:1 with one or more substitutions, wherein the domain maintains the ability to dimerize or tetramerize.

Alternatively expressed, preferred tetramerization domains according to the invention are domains comprising a sequence of amino acids 6-36 of SEQ ID NO: 1 or comprising a sequence that differs from amino acids 6-36 of SEQ ID NO: 1 by 1, 2, 3, 4, 5 or 6 alterations, wherein the domain maintains the ability to dimerize or tetramerize.

The skilled person can easily determine whether such a domain with a given substitution maintains the ability to dimerize or tetramerize by simple routine experimentation, or find such information in the literature, e.g. in J. Gencel-Augusto and G-Lozano; Genes & Development 34:1128-1146, incorporated by reference.

A preferred tetramerization domain according to the invention is a domain with an amino acid sequence that differs from the sequence of amino acids 6-36 of SEQ ID NO: 1 by 1, 2, 3, 4, 5 or 6 substitutions selected among following substitutions:
E6V, Q, K, G, D or A;
Y7S, N, H, F, D or C;
F8Y, V, S, L, I or C;
T9S, P, N or A;
L10V, I or F;
Q11R, L, K, H or E;
I12V, T, M, L or F;
R13S, P, L, H, G or C;
G14W, R or A;
R15S, P, L, H, G or C;
E16V, Q, K, G, D or A;
F18Y, V, S, L, I or C;
E19V, Q, K, G, D or A;
M20V, T, R, L, K or I;
F21L or I;
R22L or G;
E23V, Q, K, G, D or A;
L24M;
N25S, I or D;
E26V, Q, K, G, D or A;
A27V, T, S, G or D;
L28W, V, M or F;
E29Q, G or D;
L30V, R, I, H or F;
K31T, R, Q, N, M or E;
D32Y, V, N, H, G or A;
A33V, T, S, P, G or D;
Q34R, L, K, H or E;
using the numbering of SEQ ID NO: 1. All these substitutions are known in the art, and it is known that they do not abolish the domains ability to form dimers and/or tetramers.

The L24P substitution abolished tetramerization completely and should not be applied.

The p53 tetramerization domain comprising the sequence of amino acids 6-36 of SEQ ID NO: 1, is a preferred SADA domain.

The compound of the invention may further comprise one or more linkers separating the different part of the compound, e.g., separating the first binding site from the second binding site or separating the second binding site from the tetramerization domain. The purpose of the linker is to separate the different domains allowing them to fold and function without hindrance from other elements of the compound. The linker is preferably composed of hydrophilic residues that do not generate strong secondary structures, and is typically rich in residues such as glycine, serine and/or threonine. A preferred linker is a linker composed on G and S residues such as GGGGS (SEQ ID NO: 9), optionally repeated two or more times to obtain a desirable length.

In one preferred embodiment, the first antigen binding site is a VHH binding HER2, the second antigen binding site is a Fab capable of binding DOTA and the tetramerization domain is p53. The Fab may conveniently be derived from the antibody 2D12.5, optionally with the C825 substitutions disclosed in WO 2010/099536. An example of this embodiment is the compound comprising a first polypeptide comprising the sequence of SEQ ID NO: 10, and a second polypeptide comprising the sequence of SEQ ID NO: 11.

In another preferred embodiment, the first antigen binding site is a VHH binding CD38, the second antigen binding site is a Fab capable of binding DOTA and the tetramerization domain is p53. The Fab may conveniently be derived from the antibody 2D12.5, optionally with the C825 substitutions disclosed in WO 2010/099536.

An example of this embodiment is the compound comprising a first polypeptide comprising the sequence of SEQ ID NO: 12, and a second polypeptide comprising the sequence of SEQ ID NO: 11; a compound comprising a first polypeptide comprising the sequence of SEQ ID NO: 12, and a second polypeptide comprising the sequence of SEQ ID NO: 14; a compound comprising a first polypeptide comprising the sequence of SEQ ID NO: 12, and a second polypeptide comprising the sequence of SEQ ID NO: 15; a compound comprising a first polypeptide comprising the sequence of SEQ ID NO: 23, and a second polypeptide comprising the sequence of SEQ ID NO: 27; or a compound comprising a first polypeptide comprising the sequence of SEQ ID NO: 24, and a second polypeptide comprising the sequence of SEQ ID NO: 25; a compound comprising a first polypeptide comprising the sequence of SEQ ID NO: 23, and a second polypeptide comprising the sequence of SEQ ID NO: 25.

In another preferred embodiment, the first antigen binding site is a VHH binding CD38 and the second antigen binding site is a Fab capable of binding DOTA. This embodiment does not comprise the tetramerization domain p53.

An example of this embodiment is the compound comprising a first polypeptide comprising the sequence of SEQ ID NO: 12, and a second polypeptide comprising the sequence of SEQ ID NO: 13.

In another preferred embodiment, the first antigen binding site is a VHH binding CD38 and the second antigen binding site is a humanized Fab capable of binding DOTA and the tetramerization domain p53.

An example of this embodiment is the compound comprising a first polypeptide comprising the sequence of SEQ ID NO: 28, and a second polypeptide comprising the sequence of SEQ ID NO: 29.

In another preferred embodiment, the first antigen binding site is a VHH binding CD38, the second antigen binding site is a Fab capable of binding DOTAM and the tetramerization domain is p53.

An example of this embodiment is the compound comprising a first polypeptide comprising the sequence of SEQ ID NO: 16, and a second polypeptide comprising the sequence of SEQ ID NO: 17.

In another preferred embodiment, the first antigen binding site is a VHH binding CD38 and the second antigen binding site is a Fab capable of binding DOTAM. This embodiment does not comprise the tetramerization domain p53.

An example of this embodiment is the compound comprising a first polypeptide comprising the sequence of SEQ ID NO: 16, and a second polypeptide comprising the sequence of SEQ ID NO: 18.

In another preferred embodiment, the first antigen binding site is a VHH binding BAFF (B-cell activating factor, (TNF), the second antigen binding site is a Fab capable of binding DOTA and the tetramerization domain is p53. The Fab may conveniently be derived from the antibody 2D12.5, optionally with the C825 substitutions disclosed in WO 2010/099536.

An example of this embodiment is the compound comprising a first polypeptide comprising the sequence of SEQ ID NO: 19, and a second polypeptide comprising the sequence of SEQ ID NO: 27.

In another preferred embodiment, the first antigen binding site is a VHH binding CD33, the second antigen binding site is a Fab capable of binding DOTA and the tetramerization domain is p53. The Fab may conveniently be derived from the antibody 2D12.5, optionally with the C825 substitutions disclosed in WO 2010/099536.

An example of this embodiment is the compound comprising a first polypeptide comprising the sequence of SEQ ID NO: 20, and a second polypeptide comprising the sequence of SEQ ID NO: 27.

In another preferred embodiment, the first antigen binding site is a VHH binding EGF-R variant 3 (EGFRvIII), the second antigen binding site is a Fab capable of binding DOTA and the tetramerization domain is p53. The Fab may conveniently be derived from the antibody 2D12.5, optionally with the C825 substitutions disclosed in WO 2010/099536.

An example of this embodiment is the compound comprising a first polypeptide comprising the sequence of SEQ ID NO: 21, and a second polypeptide comprising the sequence of SEQ ID NO: 27.

In another preferred embodiment, the first antigen binding site is a VHH binding CEA, the second antigen binding site is a Fab capable of binding DOTA and the tetramerization domain is p53. The Fab may conveniently be derived from the antibody 2D12.5, optionally with the C825 substitutions disclosed in WO 2010/099536.

An example of this embodiment is the compound comprising a first polypeptide comprising the sequence of SEQ ID NO: 22, and a second polypeptide comprising the sequence of SEQ ID NO: 27.

In another preferred embodiment, the first antigen binding site is a VHH binding CD276, the second antigen binding site is a Fab capable of binding DOTA and the tetramerization domain is p53. The Fab may conveniently be derived from the antibody 2D12.5, optionally with the C825 substitutions disclosed in WO 2010/099536.

An example of this embodiment is the compound comprising a first polypeptide comprising the sequence of SEQ ID NO: 26, and a second polypeptide comprising the sequence of SEQ ID NO: 27.

In another preferred embodiment, the first antigen binding site is a VHH binding HER2, the second antigen binding site is a humanized Fab capable of binding DOTA and the tetramerization domain is p53. The third binding site is a VHH binding CD276. The Fab may conveniently be derived from the antibody 2D12.5, optionally with the C825 substitutions disclosed in WO 2010/099536.

An example of this embodiment is the compound comprising a first polypeptide comprising the sequence of SEQ ID NO: 30, and a second polypeptide comprising the sequence of SEQ ID NO: 31.

Another example of this embodiment is the compound comprising a first polypeptide comprising the sequence of SEQ ID NO: 30, and a second polypeptide comprising the sequence of SEQ ID NO: 32.

### Use of the Compound

The compounds of the invention are useful for immunotherapy, in particular for pretargeted radio immunotherapy (PRIT).

PRIT is used for treating cancers in methods where a bispecific antibody, comprising a first binding site capable of binding a tumor antigen and a second binding site capable of binding a radionuclide, a chelator binding a radionuclide or a molecule linked to a chelator, e.g. a peptide bound to a chelator group, where the first binding site is capable of binding the peptide part; is administered to a patient in need of treatment. After allowing the antibody to bind to the tumor and letting unbound antibody be cleared from the plasma, a radionuclide or a chelator binding a radionuclide, which radionuclide or chelator binding a radionuclide is recognized by the bispecific antibody; is administered to the patient and will be bound by the bispecific antibody localized at the tumor. Unbound radionuclide or chelator binding radionuclide will rapidly be cleared from the plasma via renal clearance.

It is preferred that the bispecific antibody is cleared from the plasma before the radionuclide is administered, in order to protect other tissues from radiation. In some embodiments, a clearing agent is administered between the administration of the bispecific antibody and the administration of radionuclide in order to improve clearance.

In one embodiment the compound of the present invention comprising a tetramerization domain are preferably administered in tetrameric form and will after administration bind to the target tumor antigen. The compound in the tetrameric form will have a higher avidity to the tumor antigen due to the coordination between the four tumor antigen binding sites of the tetrameric form, so it can be anticipated that the compound on tetrameric form will exhibit improved binding properties to the tumor due to the tetrameric format. Unbound compounds in tetrameric form will soon disintegrate into monomeric form due to the lowered concentration in plasma and the monomers will rapidly be cleared from plasma, because of their size below the renal clearance limit. These properties of the compound of the invention means that it efficiently binds to the tumor antigen, and unbound compound is rapidly cleared from plasma, so a very efficient clearance is obtained even without the use of a clearing agent.

In another embodiment, the compound of the invention comprises two identical tumor binding sites. In this embodiment the compounds will have a higher avidity to the tumor antigen due to the coordination between the two tumor antigen binding sites, compared with a similar compound containing the same binding sites, but only having one tumor binding site.

In another embodiment, the compound of the invention comprises two different tumor binding sites. In this embodiment the compounds will have improved binding properties to a tumor expressing both tumor antigens due to the coordination between the two different tumor antigen binding sites, compared with a similar compound only having one of the two tumor binding sites. One example of a tumor carrying two different tumor antigens, where it would be beneficial to use a compound according to this embodiment, is breast cancer. In some breast cancers there is overexpression of both B7H3 and HER2 on the cell surface. The breast cancer-derived cell line BT-474 can be used to address this experimentally.

Thus, in one preferred aspect the invention relates to a method of treating or diagnosing cancer in a patient, comprising the steps of
i. Administering a compound according to the invention, which compound is capable of binding a tumor antigen and further capable of binding a chelator with a bound radionuclide, to a subject in need of such treatment or diagnosis;
ii. After a holding period, administering the chelator binding a radionuclide to the subject.

In case the compound comprises a tetramerization domain the compound will disassemble into monomers and unbound compound will be cleared from plasma during the holding period.

The holding period may be selected in the range of 12 h to 7 days, e.g., 12 h, 18 h, 24 h, 36 h, 2 days, 3 days, 4, days, 5 days, 6 days, or 7 days.

Even though a satisfactory efficient clearing may be obtained using the compound of the invention, it is possible to include a step of administering a clearing agent in order to improve clearance. However, in most situations it is superfluous and desirable to omit, in order to avoid a further administration step and the inconvenience for the patient connected to such an additional step.

Similar methods of treatment have previously been disclosed in e.g., WO 2018/204873 with the significant difference that the methods of the prior art use bispecific antibody constructs based on antigen binding sites in the scFv format.

It is known that there are some inherent difficulties with the scFv format, such as low productivity, and often some heterogenicity is observed in the product, presumably caused by the mispairing of disulfide bonds, which problem may be aggravated by the frequently used practice of introducing a stabilizing disulfide bond between the variable light chain and the variable heavy chain. Further, many scFvs have low solubility, and low stability upon storage is also often observed.

Consequently, it is known that some scFv's require several rounds of amino acid alterations in order to mitigate with these inherent difficulties. On the contrary, the molecules of the invention are based on natural binding domains that do not give rise to such difficulties, or in general has fewer and less severe problems that need to be handled as part of the development of a pharmaceutical product.

The compounds of the invention are superior to the scFv based conjugates disclosed in the art, in respect of manufacturability and product stability, and provides at least the following benefits:
Higher expression levels,
Easier purification, e.g., using robust manufacturing capture purification methods,
Higher solubility,
Broad formulation options,
Build on natural domains,
Improved stability, and
Less heterogenicity.

Thus, in many ways the compounds of the invention are “better molecules” that are better suited for the preparation of pharmaceutical products, compared to scFv based compounds.

In one embodiment the cancer is selected among osteosarcoma, neuroblastoma, liposarcoma, fibrosarcoma, carcinoma, malignant fibrous histiocytoma, leiomyosarcoma, spindle cell sarcoma, brain tumor, small cell lung cancer, retinoblastoma, HTLV-1 infected T cell leukemia, breast cancer, colon cancer, prostate cancer, T-cell and B-cell lymphomas, glioblastoma multiforme, malignant glioma, Head and Neck cancer, solid tumors and non-small-cell lung cancer.

The skilled person will appreciate that the chelator used in the method of treatment/diagnosis of the invention can be any chelator that is recognized and can be bound by the second antigen binding site.

In one preferred embodiment, the second antigen binding site is capable of binding DOTA, or a derivative of DOTA. In this embodiment the chelator is DOTA or a derivative of DOTA such as the compounds comprising a DOTA ring system, to which the second antigen binding site can bind, e.g., compounds disclosed in WO 2010/099536, WO2019/010299 and WO 2022/005998 (incorporated herein by reference).

The radionuclide may be any radionuclide that can be bound by a chelator, typically radionuclide cations. Examples of suitable radionuclides include: ²²⁵Ac, ²²⁷Ac, ²⁴¹Am, ²¹¹At, ²¹⁵At, ²¹⁷At, ²¹⁸At, ²⁰⁹Bi, ²¹¹Bi, ²¹²Bi, ²¹³Bi, ²⁴⁹Cf, ²⁵²Cf, ²⁴⁴Cm, ²⁴⁵Cm, ²⁴⁸Cm, ⁵⁷Co, ⁵⁸Co, ⁵¹Cr, ⁶⁴Cu, ⁶⁷Cu, ¹⁵²Dy, ¹⁶⁵Dy, ¹⁵²Eu, ⁵⁹Fe, ²²¹Fr, ⁶⁷Ga, ⁶⁸Ga, ⁶⁶Ga, ¹⁶¹Ho, ^{110m}In, ¹¹¹In, ¹⁹²Ir, ¹³³La, ¹⁷⁷Lu, ²³⁷Np, ^{189m}Os, ²³¹Pa, ²⁰³Pb, ²¹²Pb, ²¹⁰Po, ²¹¹Po, ²¹²Po, ²¹⁴Po, ²¹⁵Po, ²¹⁶Po, ²¹⁸Po, ^{195m}Pt, ²³⁸Pu, ²³⁹Pu, ²⁴⁰Pu, ²⁴⁴Pu, ²²³Ra, ²²⁴Ra, ²²⁶Ra, ⁸²Rb, ¹⁸⁶Re, ¹⁸⁸Re, ^{103m}Rh, ¹¹⁹Sb, ⁷⁵Se, ⁸⁹Sr, ¹⁴⁹Tb, ¹⁵¹Tb, ¹⁶¹Tb ^{99m}Tc, ^{94m}Tc, ²²⁷Th, ²²⁸Th, ²²⁹Th, ²³⁰Th, ²³²Th, ²⁰¹Tl, ⁹⁰Y, ⁸⁶Y, ⁸⁹Zr.

In one embodiment the method of the invention is a method for treating cancer. In this embodiment the skilled person will select a suitable radionuclide e.g., a radionuclide delivering a high energy, and preferably with a limited penetration so only the targeted tissue is affected. The treating physician will be able to select a suitable radionuclide for treatment without exercising any inventive activity.

In one embodiment, the method of the invention is a method for diagnosing cancer. In this embodiment, the method typically comprises a subsequent step of detecting the radionuclides bound to the compound of the invention and localized at the surface of tumor cells. In this embodiment, the skilled person may select a radionuclide having a long penetration range and which deposits a low energy in surrounding tissues. It is within the skills of the average practitioner to select a suitable radionuclide for the diagnosis. The detection may be performed using well known methods and equipment for detecting radionuclides, such as a PET or SPECT scanner.

In some embodiments the method of the invention comprises a second and optional subsequent step(s) of an administration of chelator with bound radionuclide. Such a second administration of chelator with bound radionuclide is typically done 1-7 days after the first administration of chelator with bound radionuclide, and a subsequent administration of chelator with bound radionuclide is typically done 1-7 days after the previous administration of chelator with bound radionuclide. The radionuclide and/or chelator administered in the first, second and optional subsequent administration may be the same or it may be a different radionuclide and/or chelator. For example, an alpha-emitter may be administered in the first administration and a beta-emitter administered in the second and optional subsequent administration. Or in another example, a radionuclide suitable for PET or SPECT scanning is administered in the first administration and a scanning is performed in order to detect the tumor(s), and a radionuclide more suited to eradicate the tumor cells is administered in the second and optional subsequent administration.

### Nucleic Acid Sequences Etc.

The invention also relates to nucleic acid sequences encoding the compound of the invention. The nucleic acids may be provided by methods known in the art, e.g., starting from nucleic acid sequences encoding the separate elements of the compound, assembling and modifying the sequences using methods known in the art.

Alternatively, the nucleic acids sequences may be obtained by DNA synthesis, for example, by designing the amino acid sequences for the intended compound, deriving a suitable nucleic acid sequence encoding the intended amino acid sequence and synthesizing the sequence using methods known in the state of the art. This method has the benefit that it is easy to adapt the codon usage to the intended host cell and also to provide the nucleic acid with suitable sequences required for expression in the intended host cell, such as promoters, RBS, Kozak sequence, terminator, polyadenylation site etc. This is all within the skills of the average practitioner to design a suitable nucleic acid sequence encoding the intended amino acid sequence once the intended amino acid sequence has been designed.

### Production of Molecules

The nucleic acid sequences encoding the compound of the invention may be inserted into an expression construct, such as an expression vector, transformed into a selected host cell and expressed, leading to formation of the compound.

In the embodiment where the first and the second antigen binding sites are VHH fragments, the complete nucleic acid sequence encoding the compound can be contained in a single expression construct for production as a single polypeptide chain.

In the embodiment where one of the first and the second antigen binding sites is a VHH and the other is a Fab fragment, the skilled person will appreciate that two expression constructs are necessary, one for each strand of the compound. The two expression constructs may be inserted and expressed in the same cell. The two chains can be expressed in one cell either by transfection with two vectors or by one vector having bicistronic expression sites.

A suitable host cell for use according to the invention may in principle be any host cell capable of expressing the polypeptides of the compound. Such host cells and expression systems suitable for particular polypeptides are known in the art and selecting a suitable expression system for a particular compound, including expression vectors and host cells, are within the skills of the average practitioner.

Examples of suitable host cells include bacterial cells, such as E. coli, Bacillus sp., such as B licheniformis and B. subtilis; fungal cells such as Saccharomyces cerevisiae, Pichia pastoris, Aspergillus niger, A. oryzae, Trichoderma reesei, Pencillium chrysogenum; insect cells, Mammalian cells such as Hela cells, CHO cells, HEK cells.

Mammalian cells such as Hela cells, CHO cells and HEK cells are preferred because it is well known that these cells can not only produce the two polypeptide chains, but they can also combine the two chains correctly and produce the complete molecule of the invention.

In order to produce compounds of the invention wherein one of the first and the second antigen binding sites is a VHH and the other is a Fab fragment, it is advantageous to produce the compound using basically same technology as known in the art for producing recombinant antibodies. Using this approach, the two nucleic acids encoding the two strands of the compounds are provided with suitable expression signals and transformed into same host cell. When the two nucleic acids are expressed and the two strands are formed, they will assemble in vivo and be secreted from the host cell as a single protein product even though it consists of and are expressed as two separate polypeptide strands. It has turned out that this method is highly effective for producing compounds of the invention in high purity and yields.

After production the compound of the invention is recovered from the cell culture supernatant using methods known in the art, such as precipitation, affinity purification and chromatographic methods. For compounds of the invention comprising a Fab fragment, same recovery methods as used for complete antibodies may conveniently be used, such as Protein A or Protein L affinity purification.

### Compositions

The invention also relates to compositions comprising one or more compounds of the invention.

The compositions comprise in addition to the compound of the invention, one or more of diluents, salts, pH regulating agents, stabilizers, antioxidants, tonicity regulating agents etc.

In a preferred embodiment, the composition is a pharmaceutical composition comprising only pharmaceutically acceptable ingredients, such as ingredients disclosed in well recognized Pharmacopoeias, e.g., as described in European Pharmacopoeia 10^{th} Edition; using methods and technologies known in the pharmaceutical or apothecary area.

All cited references are incorporated by reference.

The accompanying Figures and Examples are provided to explain rather than limit the present invention. It will be clear to the person skilled in the art that aspects, embodiments, claims and any items of the present invention may be combined.

Unless otherwise mentioned, all percentages are in weight/weight. Unless otherwise mentioned, all measurements are conducted under standard conditions (ambient temperature and pressure). Unless otherwise mentioned, test conditions are according to European Pharmacopoeia 10^{th} Edition.

### FIGURES

### Short Description of the Figures

FIG. 1A. Shows a schematic representation of a compound of the invention, comprising a VHH comprising a first binding site linked to a Fab comprising a second binding site with an optional C-terminal p53 tetramerization domain. In this example the compound consists of two polypeptide chains attached to each other via a disulfide bound localized in the constant regions of the Fab. One chain comprises the VHH binding fragment, and either the light or the heavy chain of the Fab, the other chain comprises the remaining chain (light or heavy) of the Fab and optionally the p53 tetramerization domain.

FIG. 1B. Shows a schematic representation of a compound of the invention, comprising a first VHH comprising a first binding site linked to a second VHH comprising a second binding site with an optional C-terminal p53 tetramerization domain. In this example the compound consists of a single polypeptide chain, comprising the two binding sites and optionally the tetramerization domain.

FIG. 1C. Shows a schematic representation of a compound of the invention, comprising 2 VHHs comprising a first binding site linked to a Fab comprising a second binding site with an optional C-terminal p53 tetramerization domain. In this example the compound consists of two polypeptide chains being attached to each other via a disulfide bound localized in the constant regions of the Fab. One chain comprises a first VHH binding fragment and either the light or the heavy chain of the Fab, the other chain comprises a second VHH binding fragment identical to the first VHH and the remaining chain (light or heavy) of the Fab and optionally the p53 tetramerization domain.

FIG. 1D. Shows a schematic representation of a compound of the invention, comprising 2 different VHHs each comprising a binding site different from the other, linked to a Fab comprising a second binding site with an optional C-terminal p53 tetramerization domain. In this example the compound consists of two polypeptide chains being attached to each other via a disulfide bound localized in the constant regions of the Fab. This compound is a trispecific YPRIT. One chain comprises a first VHH binding fragment and either the light or the heavy chain of the Fab, the other chain comprises a second VHH binding fragment different from the first VHH and the remaining chain (light or heavy) of the Fab and optionally the p53 tetramerization domain.

FIG. 1E. Shows a schematic representation of a compound of the invention, comprising a VHH comprising a first binding site linked to a Fab comprising a second binding site with an optional C-terminal p53 tetramerization domain. In this example the compound consists of two polypeptide chains being attached to each other via a disulfide bound localized in the constant regions of the Fab. One chain comprises either the light or the heavy chain of the Fab and a C-terminal VHH binding fragment. The other chain comprises the remaining chain (light or heavy) of the Fab and optionally the p53 tetramerization domain.

FIG. 1F. Shows a schematic representation of a compound of the invention, comprising 2 identical VHHs comprising a first binding site linked to a Fab comprising a second binding site with an optional C-terminal p53 tetramerization domain. In this example the compound consists of two polypeptide chains being attached to each other via a disulfide bound localized in the constant regions of the Fab. This compound is bivalent on a first antigen binding site. One chain comprises either the light or the heavy chain of the Fab and a C-terminal first VHH binding fragment. The other chain comprises the remaining chain (light or heavy) of the Fab, a C-terminal second VHH identical to the first VHH fragment and optionally the p53 tetramerization domain.

FIG. 1G. Shows a schematic representation of a compound of the invention, comprising 2 different VHHs each comprising a binding site different from the other, linked to a Fab comprising a second binding site with an optional C-terminal p53 tetramerization domain. In this example the compound consists of two polypeptide chains being attached to each other via a disulfide bound localized in the constant regions of the Fab. This compound is a trispecific YPRIT. One chain comprises either the light or the heavy chain of the Fab and a C-terminal first VHH binding fragment. The other chain comprises the remaining chain (light or heavy) of the Fab, a C-terminal second VHH different from the first VHH fragment and optionally the p53 tetramerization domain.

FIG. 2A. HPLC-SEC analysis of HER2-DOTA (fab)-p53. For further details see example 2.

FIG. 2B. HPLC-SEC analysis of CD38-DOTA (fab)-p53. For further details see example 2.

FIG. 3A. HPLC-SEC analysis of CD38-DOTA (fab)-p53_C-TERM-LC. For further details see example 3.

FIG. 3B. HPLC-SEC analysis of CD33-DOTA (fab)-p53. For further details see example 3.

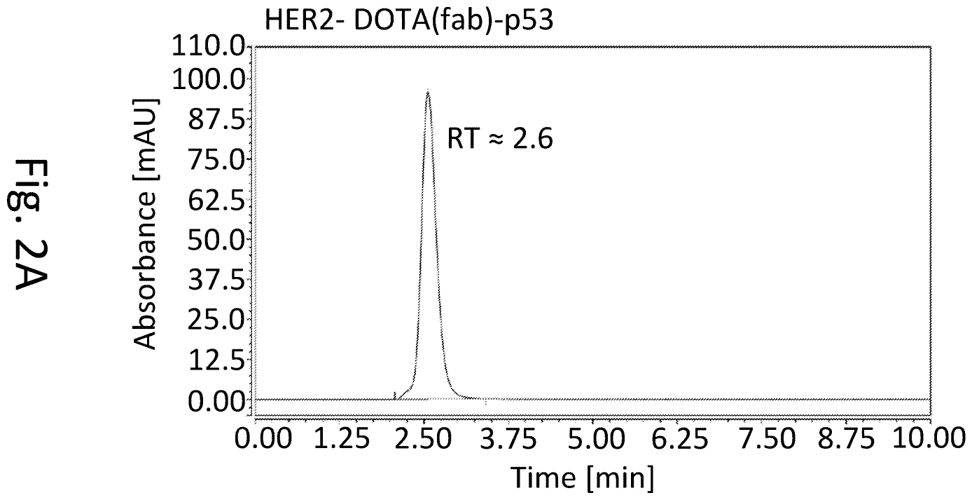

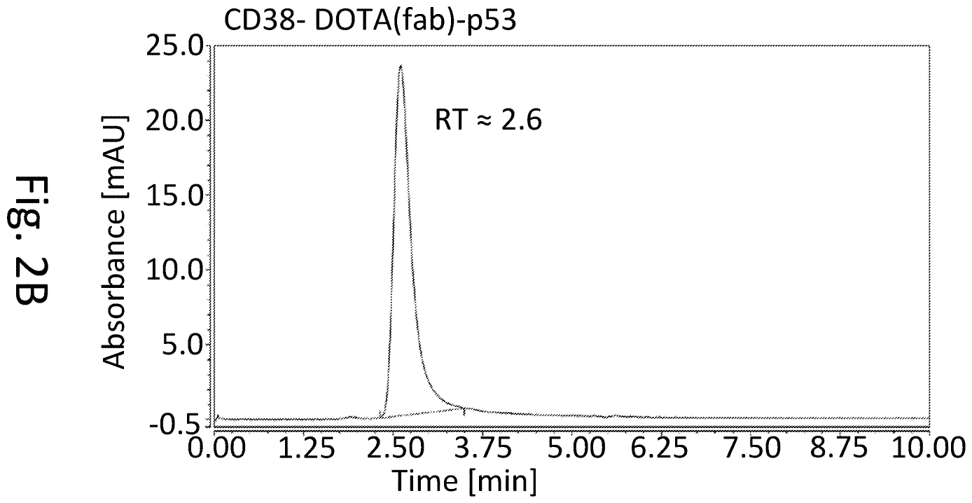

FIG. 4. HPLC-SEC analysis of CD38-DOTA (fab)-dp53—For further details see example 4

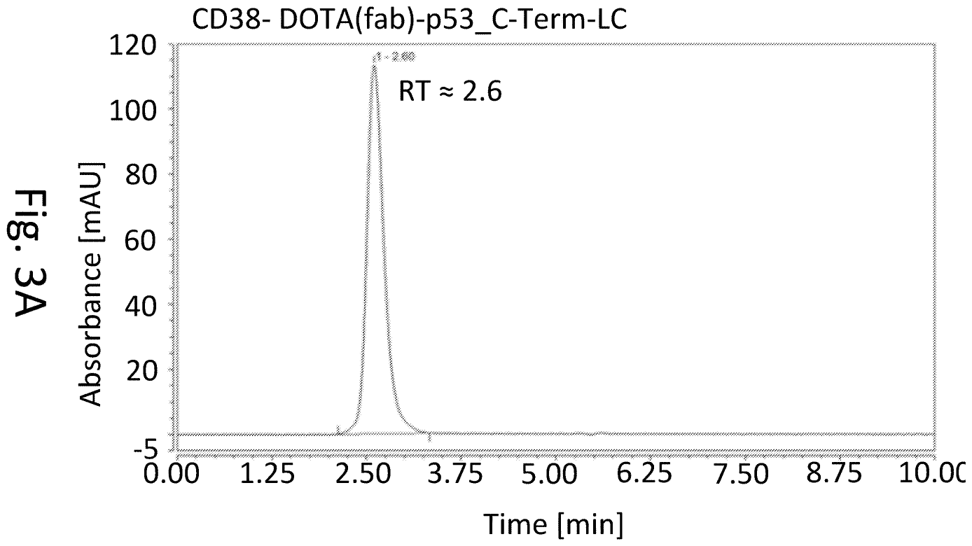

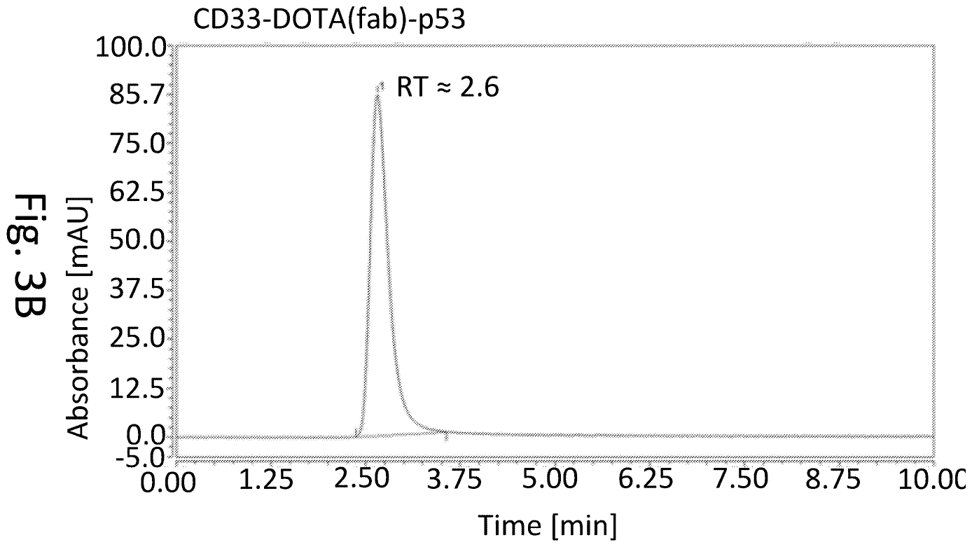

FIG. 5. Dynamic light scattering (DLS) results for CD38-DOTA (fab)-p53 and HER2-DOTA (fab)-p53. For further details see example 5.

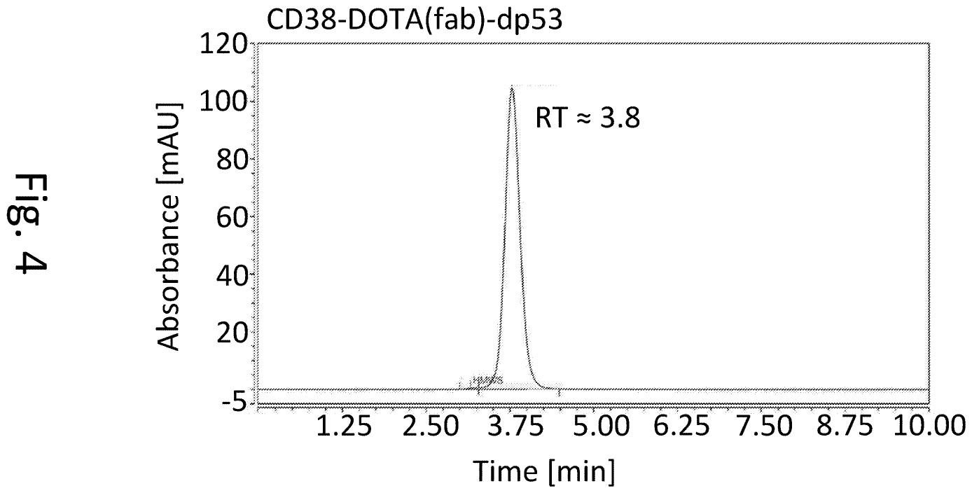

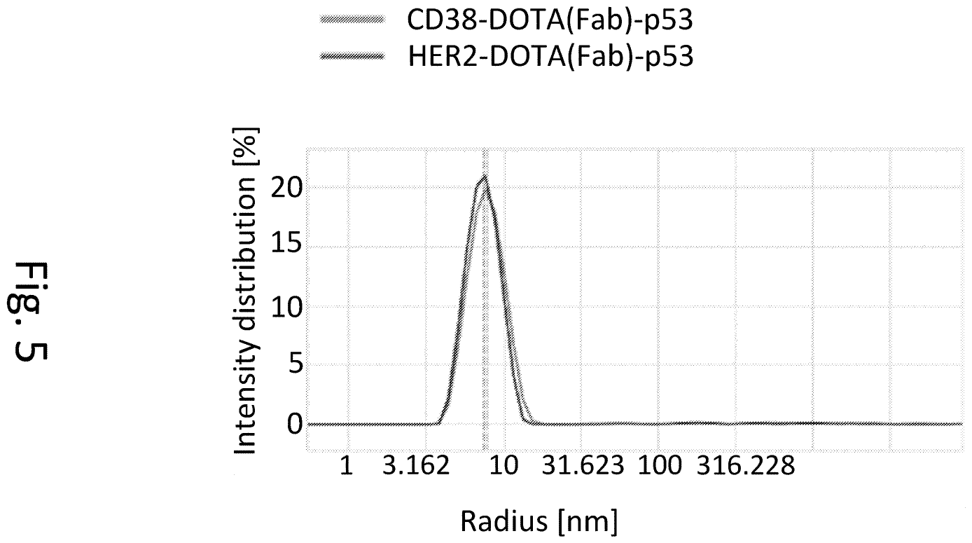

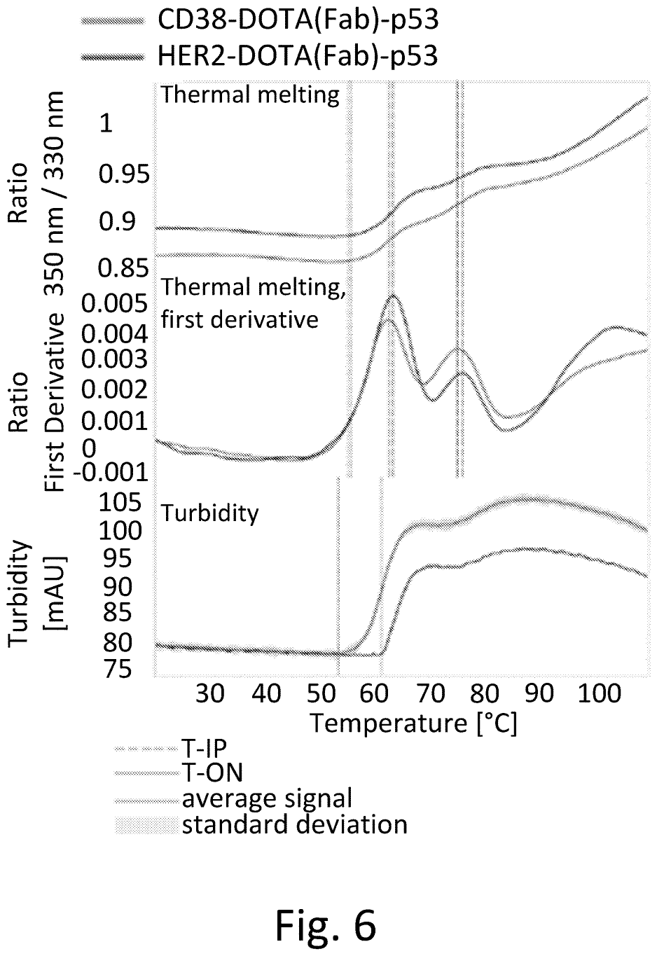

FIG. 6. Nano Differential Scanning Flourimetry (DSF) results for CD38-DOTA (fab)-p53 and HER2-DOTA (fab)-p53. For further details see example 7.

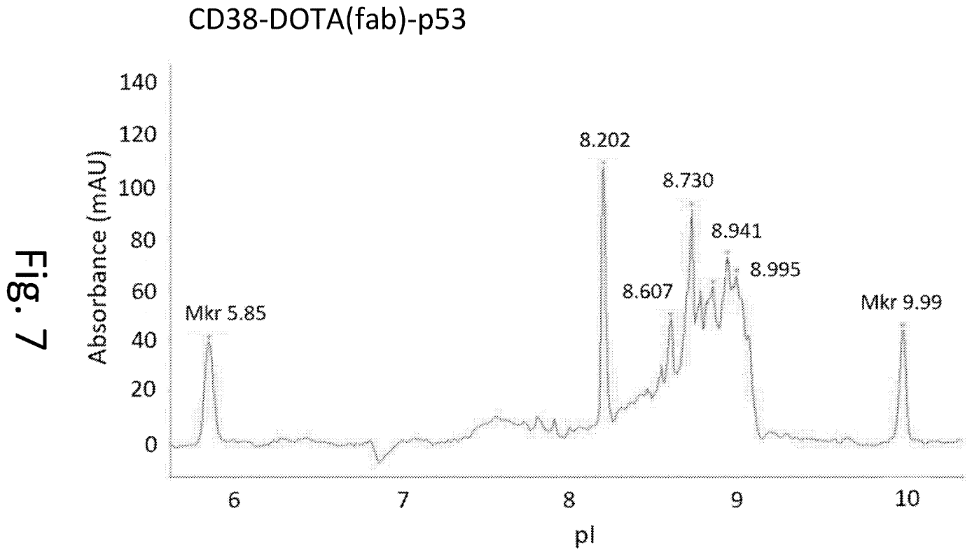

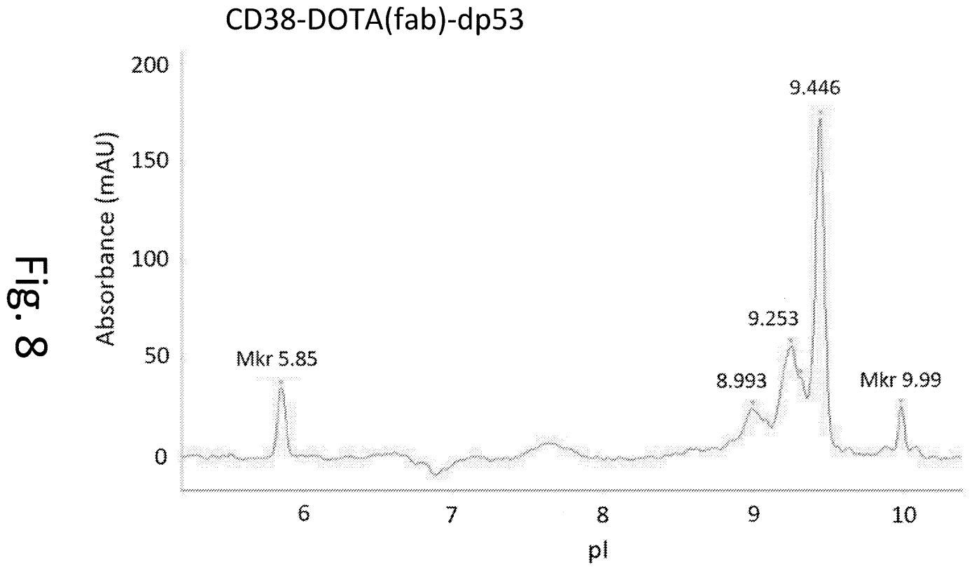

FIG. 7 and FIG. 8. Capillary isoelectric focusing (cIEF) profiles of the bispecific compounds CD38-DOTA (fab)-p53 and CD38-DOTA (fab)-dp53. For further details see example 12.

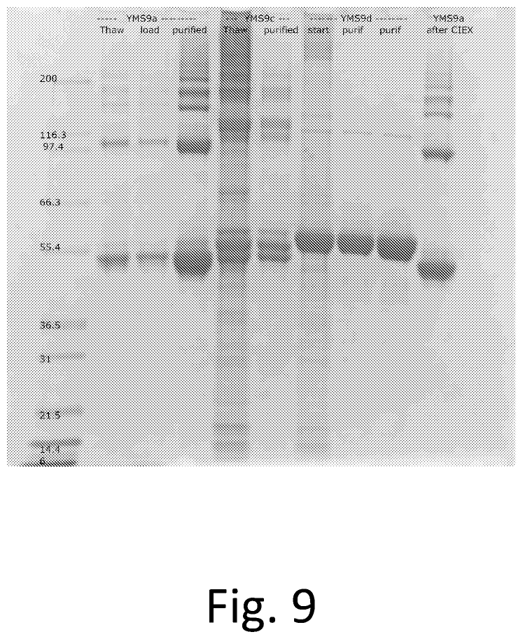

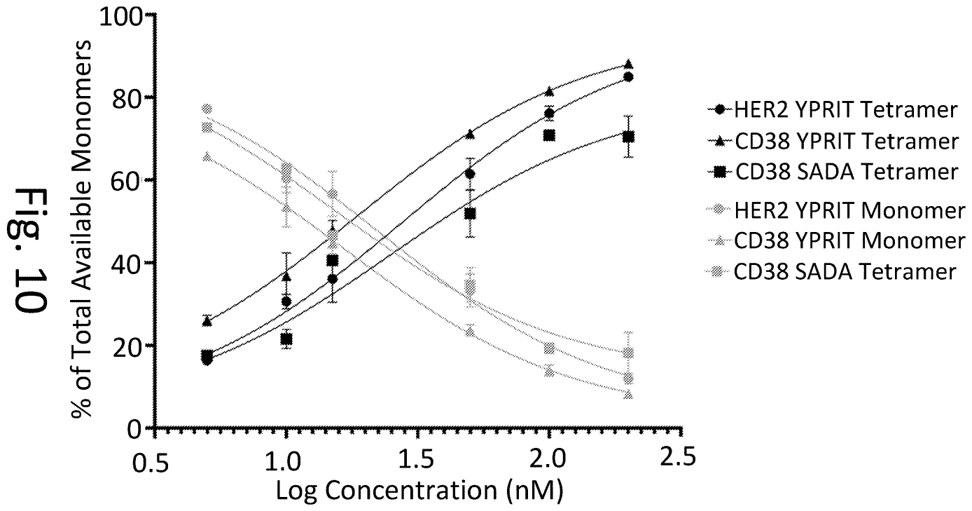

FIG. 9. YMS9a, YMS9c and YMS9d analysed on non-reducing SDS-PAGE. For further details see reference example 1.

FIG. 10. Shows tetramerization of CD38-DOTA (fab)-p53 and HER2-DOTA (fab)-p53. For further details see example 13.

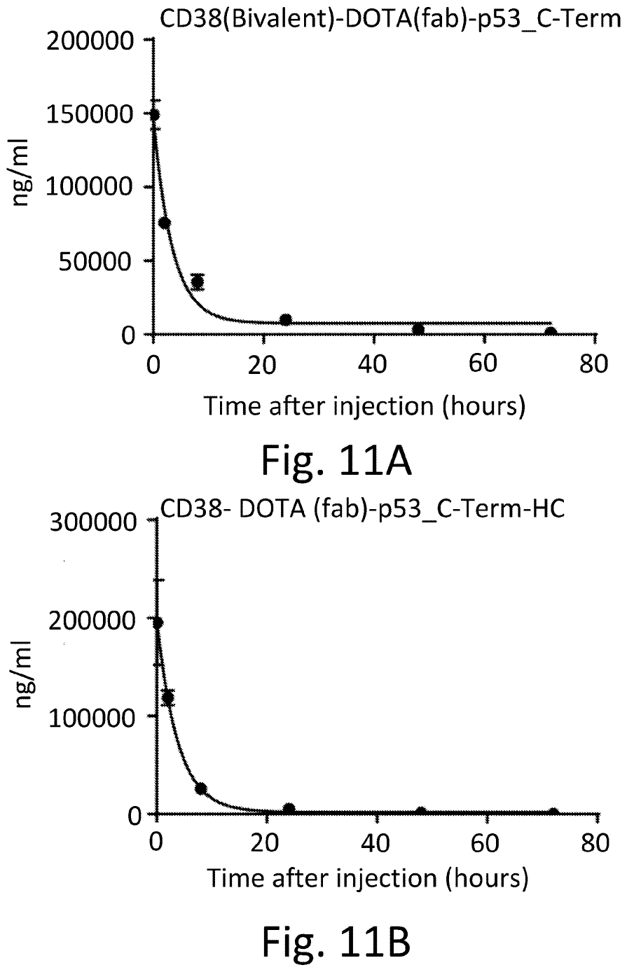

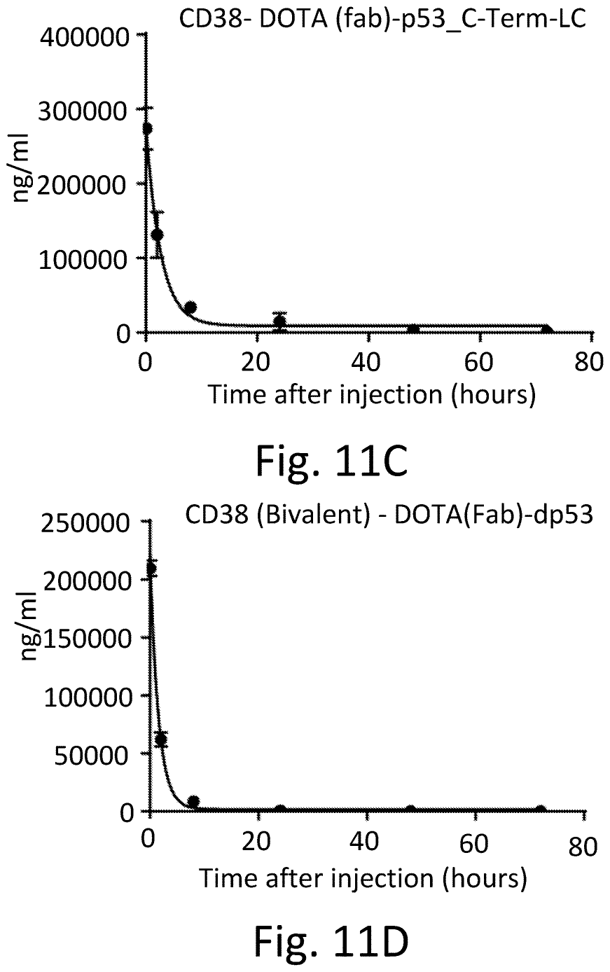

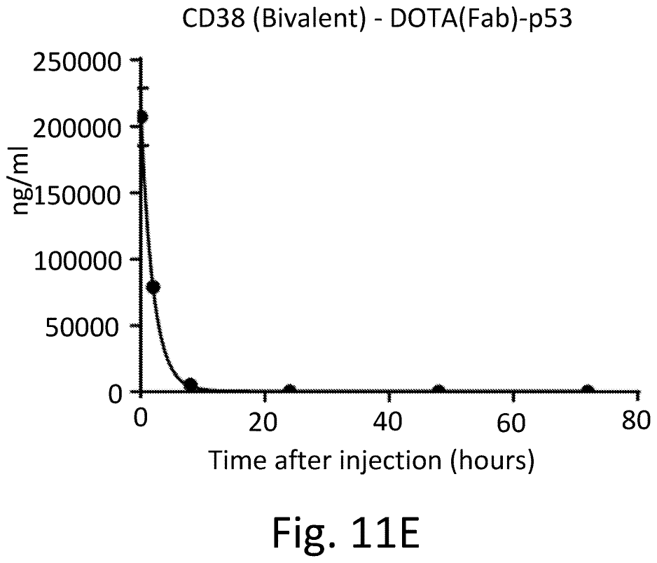

FIG. 11A. Shows pk curve for CD38 (Bivalent)-DOTA (fab)-p53_C-Term. For further details see example 14.

FIG. 11B. Shows pk curve for CD38-DOTA (fab)-p53_C-Term-HC. For further details see example 14.

FIG. 11C. Shows pk curve for CD38-DOTA (fab)-p53_C-Term-LC. For further details see example 14.

FIG. 11D. Shows pk curve for CD38 (Bivalent)-DOTA (fab)-dp53. For further details see example 14.

FIG. 11E. Shows pk curve for CD38 (bivalent)-DOTA (Fab)-p53. For further details see example 14.

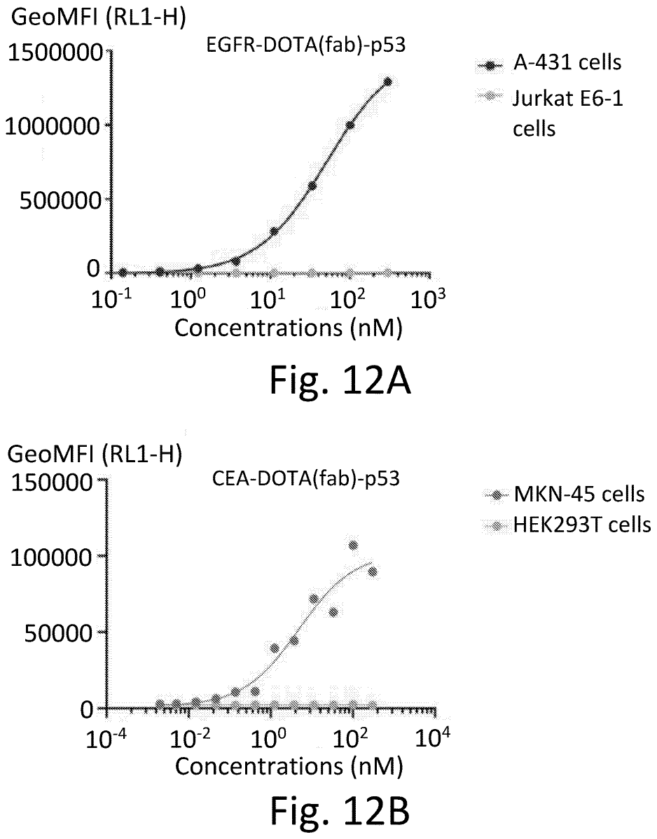

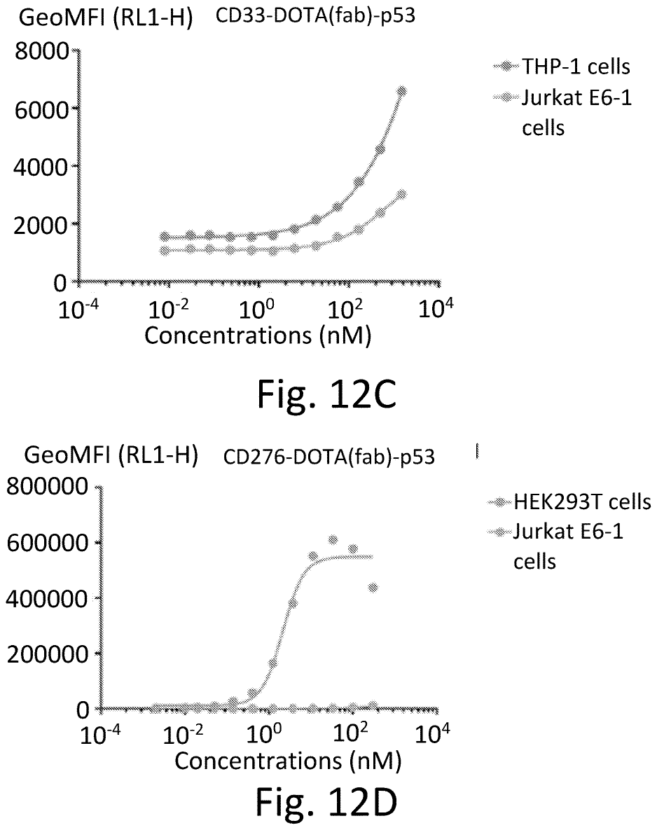

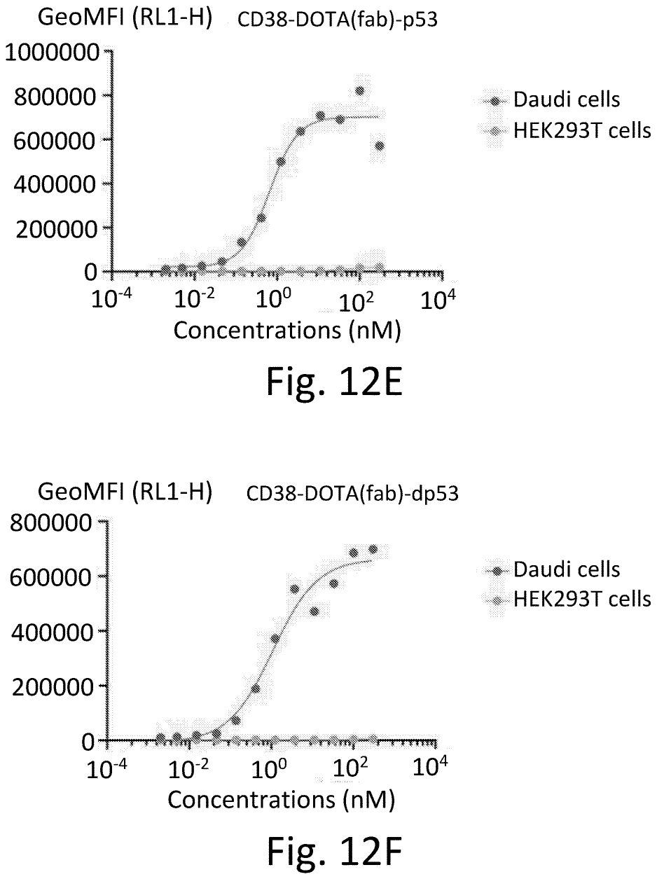

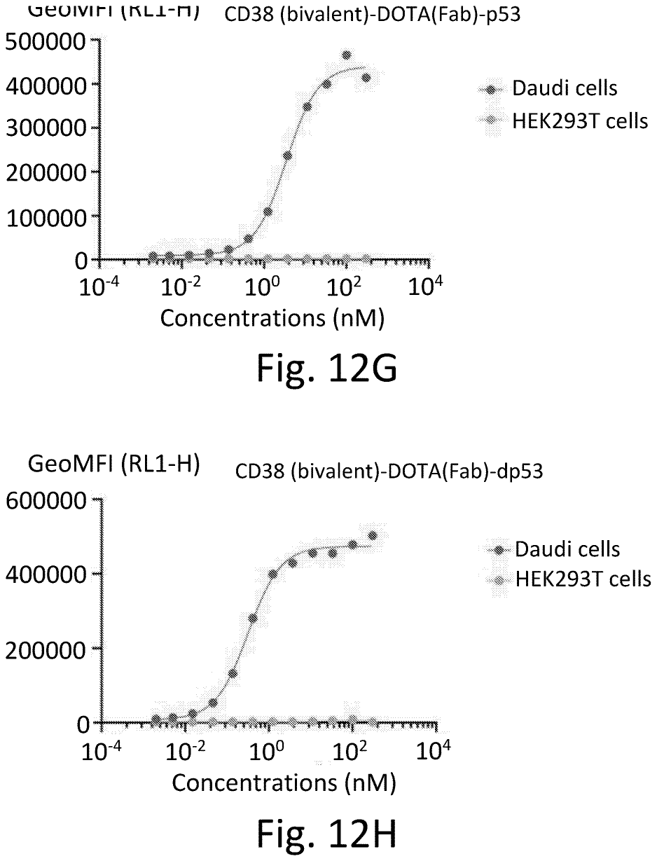

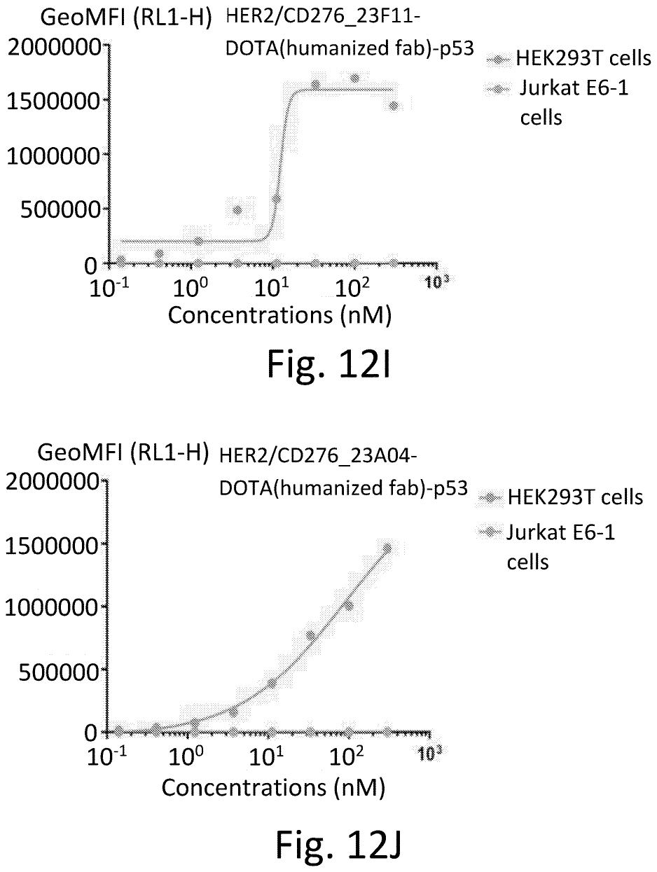

FIG. 12A. Shows EC50 determination as graphical representation of YPRIT molecules binding titration curves to target expressed in cell line for EGFR-DOTA (fab)-p53. For further details see example 15.

FIG. 12B. Shows EC50 determination as graphical representation of YPRIT molecules binding titration curves to target expressed in cell line for CEA-DOTA (fab)-p53. For further details see example 15.

FIG. 12C. Shows EC50 determination as graphical representation of YPRIT molecules binding titration curves to target expressed in cell line for CD33-DOTA (fab)-p53. For further details see example 15.

FIG. 12D. Shows EC50 determination as graphical representation of YPRIT molecules binding titration curves to target expressed in cell line for CD276-DOTA (fab)-p53. For further details see example 15.

FIG. 12E. Shows EC50 determination as graphical representation of YPRIT molecules binding titration curves to target expressed in cell line for CD38-DOTA (fab)-p53. For further details see example 15.

FIG. 12F. Shows EC50 determination as graphical representation of YPRIT molecules binding titration curves to target expressed in cell line for CD38-DOTA (fab)-dp53. For further details see example 15.

FIG. 12G. Shows EC50 determination as graphical representation of YPRIT molecules binding titration curves to target expressed in cell line for CD38 (bivalent)-DOTA (Fab)-p53. For further details see example 15.

FIG. 12H. Shows EC50 determination as graphical representation of YPRIT molecules binding titration curves to target expressed in cell line for CD38 (bivalent)-DOTA (Fab)-dp53. For further details see example 15.

FIG. 12I. Shows EC50 determination as graphical representation of YPRIT molecules binding titration curves to target expressed in cell line for HER2/CD276_23F11-DOTA (humanized fab)-p53. For further details see example 15.

FIG. 12J. Shows EC50 determination as graphical representation of YPRIT molecules binding titration curves to target expressed in cell line for HER2/CD276_23F11-DOTA (humanized fab)-p53. For further details see example 15.

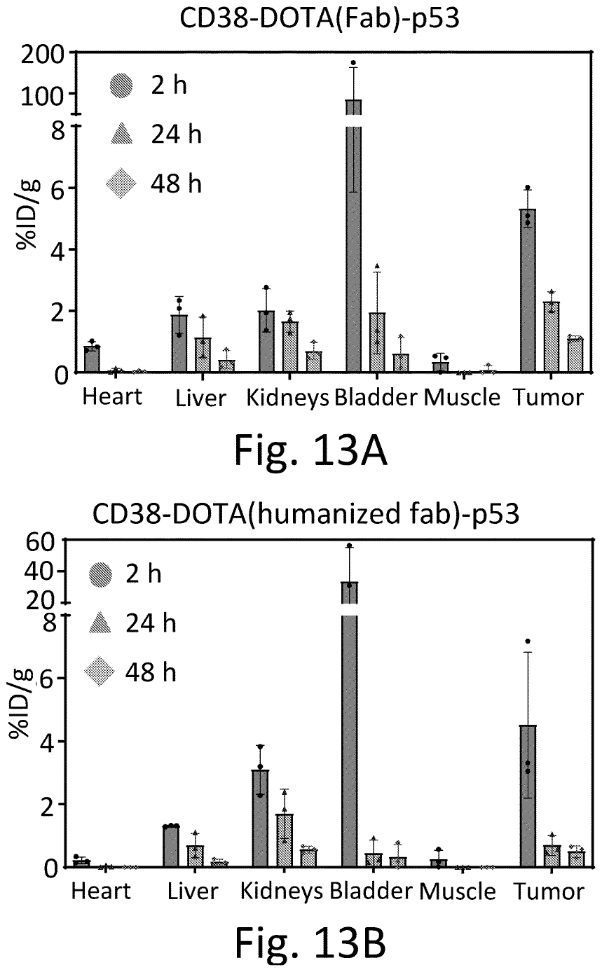

FIG. 13A. Shows in vivo distribution of CD38-DOTA (Fab)-p53 assessed by SPECT-based quantification. For further details see example 16.

FIG. 13B. Shows in vivo distribution of CD38-DOTA (humanized fab)-p53 assessed by SPECT-based quantification. For further details see example 16.

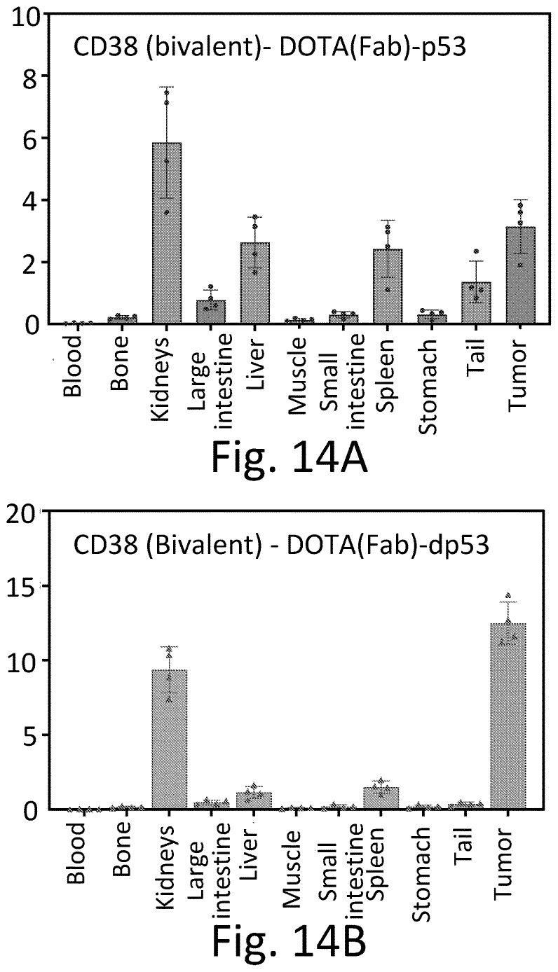

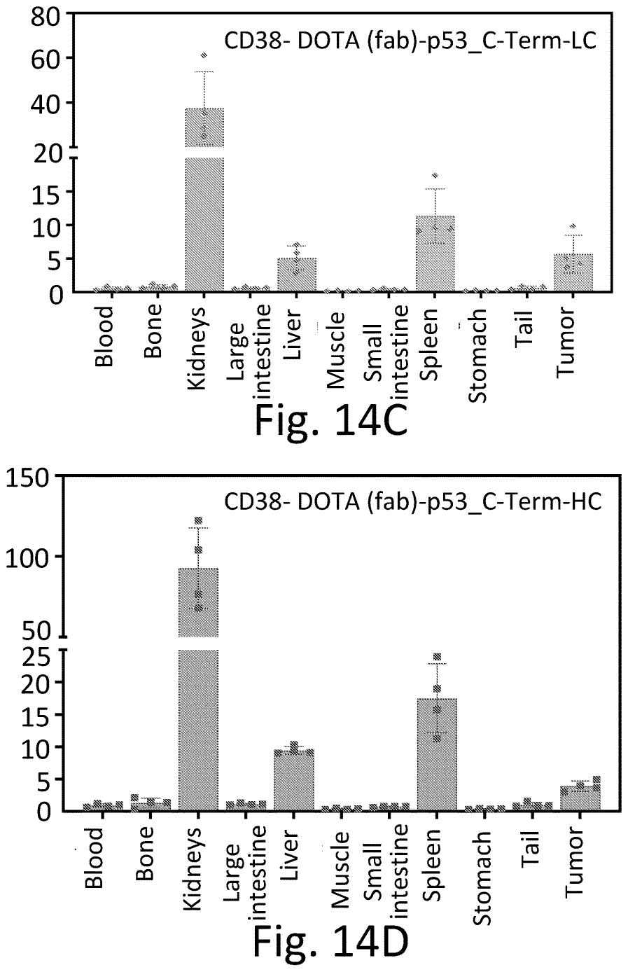

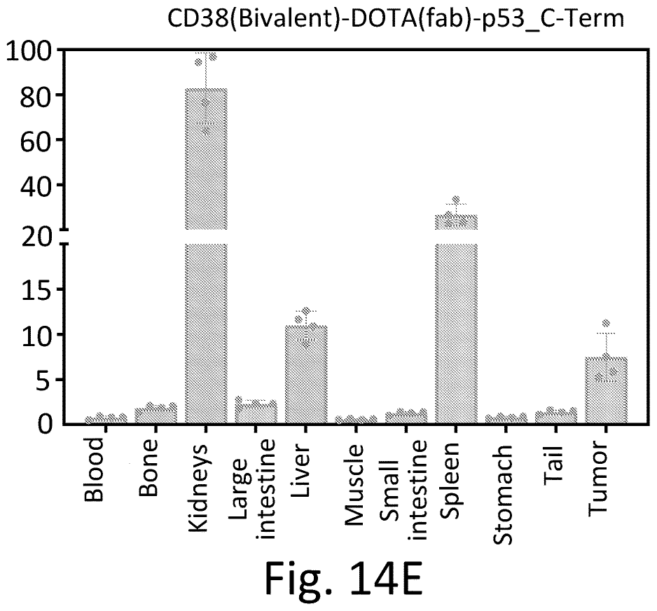

FIG. 14A shows biodistribution of CD38 (bivalent)-DOTA (Fab)-p53

FIG. 14B shows biodistribution of CD38 (Bivalent)-DOTA (fab)-dp53

FIG. 14C shows biodistribution of CD38-DOTA (fab)-p53_C-Term-LC

FIG. 14D shows biodistribution of CD38-DOTA (fab)-p53_C-Term-HC

FIG. 14E shows biodistribution of CD38 (Bivalent)-DOTA (fab)-p53_C-Term

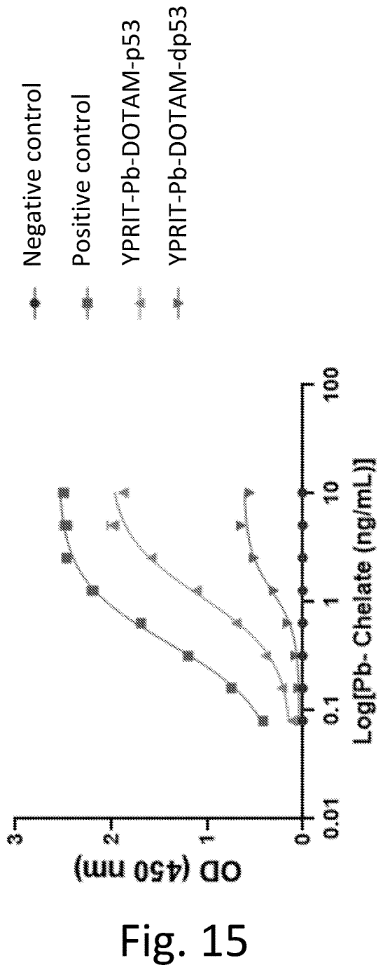

FIG. 15 shows YPRIT binding to Pb-TCMC-PEG4-Biotin (Biotinylated variant of Pb-DOTAM/Pb-TCMC.

### SHORT DESCRIPTION OF THE SEQUENCES

SEQ ID NO 1: shows the amino acid sequence comprising the P53 tetramerization domain. The p53 tetramerization domain consists of amino acids 6 to 36 of the sequence;
SEQ ID NO 2: shows the amino acid sequence of the P63 tetramerization domain;
SEQ ID NO 3: shows the amino acid sequence of the P73 tetramerization domain;
SEQ ID NO 4: shows the amino acid sequence of the hnRNPC tetramerization domain;
SEQ ID NO 5: shows the amino acid sequence of the SNAP23 tetramerization domain;
SEQ ID NO 6: shows the amino acid sequence of the StefinB tetramerization domain;
SEQ ID NO 7: shows the amino acid sequence of the KCNQ4 tetramerization domain;
SEQ ID NO 8: shows the amino acid sequence of the CBFA2T1 tetramerization domain;
SEQ ID NO: 9: shows the amino acid sequence of the G4S linker;
SEQ ID NO: 10: shows the amino acid sequence of one polypeptide chain of the HER2-DOTA (Fab)-p53 compound disclosed in example 1. Amino acids 1-115 is a HER2 binding VHH fragment, 116-132 is G4S linker, and 133-347 is the light chain of a DOTA binding Fab.
SEQ ID NO: 11: shows the amino acid sequence of the other polypeptide chain of the YPRIT compound disclosed in Example 1. Amino acids 1-222 is the heavy chain of the DOTA binding Fab, 223-232 is G4S linker, 238-268 is the p53 tetramerization domain, and 279-284 is His-tag.

Same polypeptide chain was used for the CD38-DOTA (Fab)-p53 compound.
SEQ ID NO: 12: shows the amino acid sequence of one of the polypeptide chains of the CD38-DOTA (Fab)-p53 compound disclosed in Example 1. Amino acids 1-124 is a VHH fragment binding CD38, 125-145 is G4S linker, and 146-360 is the light chain of a DOTA binding Fab.
SEQ ID NO: 13: shows the amino acid sequence of a heavy chain for CD38-DOTA (fab)-dp53. Amino acids 1-222 is the heavy chain of a DOTA binding Fab, 225-230 is His-tag.
SEQ ID NO: 14: shows the amino acid sequence of a heavy chain for CD38 (bivalent)-DOTA (Fab)-p53. Amino acids 1-124 is a VHH fragment binding CD38, 125-145 is G4S linker, 146-367 is the heavy chain of a Fab binding DOTA, 368-377 is G4S linker, and 383-413 is p53 tetramerization domain.
SEQ ID NO: 15: shows the amino acid sequence of a heavy chain for CD38 (Bivalent)-DOTA (fab)-dp53. Amino acids 1-124 is a VHH fragment binding CD38, 125-145 is G4S linker, 146-368 is the heavy chain of a DOTA binding Fab.
SEQ ID NO: 16: shows the amino acid sequence of a light chain for YPRIT_CD38_DOTAM_p53. Amino acids 1-124 is a VHH fragment binding CD38, 125-145 is G4S linker, 146-364 is the light chain of a DOTAM binding Fab.
SEQ ID NO: 17: shows the amino acid sequence of a heavy chain for CD38-DOTAM (fab)-p53. Amino acids 1-224 is a heavy chain of a DOTAM binding Fab, 225-234 is G4S linker, and 240-270 is the p53 tetramerization domain.
SEQ ID NO: 18: shows the amino acid sequence of a heavy chain for CD38-DOTAM (fab)-dp53. Amino acids 1-225 is a heavy chain of a DOTAM binding Fab.
SEQ ID NO: 19: shows the amino acid sequence of a light chain for BAFF-DOTA (fab)-p53. Amino acids 1-115 is a VHH fragment binding BAFF, 116-136 is G4S linker, and 137-351 is the light chain of a DOTA binding Fab.
SEQ ID NO: 20: shows the amino acid sequence of a light chain for CD33-DOTA (fab)-p53. Amino acids 1-126 is a VHH fragment binding CD33 (22), 127-147 is G4S linker, and 148-362 is the light chain of a DOTA binding Fab.
SEQ ID NO: 21: shows the amino acid sequence of a light chain for EGFR-DOTA (fab)-p53. Amino acids 1-120 is a VHH fragment binding EGFR, 121-141 is G4S linker, and 142-356 is the light chain of a DOTA binding Fab.
SEQ ID NO: 22: shows the amino acid sequence of a light chain for CEA-DOTA (fab)-p53. Amino acids 1-120 is a VHH fragment binding CEA, 121-141 is G4S linker, and 142-356 is the light chain of a DOTA binding Fab.
SEQ ID NO: 23: shows the amino acid sequence of a light chain for a monovalent CD38-DOTA (fab)-p53_C-Term-LC. Amino acids 1-215 is the light chain of a DOTA binding Fab, 216-236 is G4S linker, and 237-360 is a VHH fragment binding CD38.
SEQ ID NO: 24: shows the amino acid sequence of a light chain for a monovalent CD38-DOTA (fab)-p53_C-Term-HC.
SEQ ID NO: 25: shows the amino acid sequence of a heavy chain for a monovalent CD38-DOTA (fab)-p53_C-Term-HC. Amino acids 1-222 is the heavy chain of a DOTA binding Fab, 223-243 is G4S linker, 244-367 is a VHH fragment binding CD38, 368-377 is G4S linker, and 383-413 is p53.
SEQ ID NO: 26: shows the amino acid sequence of a light chain for CD276-DOTA (fab)-p53. Amino acids 1-117 is a VHH fragment binding CD276 (G8), 118-137 is G4S linker, and 138-352 is the light chain of a DOTA binding Fab.
SEQ ID NO: 27: shows the amino acid sequence of a heavy chain for a YPRIT compound disclosed in Example 1 without a His-tag (6×His). Amino acids 1-222 is the heavy chain of the DOTA binding Fab, 223-232 is G4S linker, and 238-268 is the p53 tetramerization domain.
SEQ ID NO: 28: shows the amino acid sequence of a LC sequence of an CD38-DOTA (humanized fab)-p53
SEQ ID NO: 29: shows the amino acid sequence of a HC sequence of an CD38-DOTA (humanized fab)-p53
SEQ ID NO: 30: shows the amino acid sequence of a LC sequence of an HER2-DOTA (humanized fab)-p53
SEQ ID NO: 31: shows the amino acid sequence of a HC sequence of an HER2/CD276_23F11-DOTA (humanized fab)-p53
SEQ ID NO: 32: shows the amino acid sequence of a HC sequence of an HER2/CD276_23A04-DOTA (humanized fab)-p53

### Materials and Methods

### Antibodies and Antibody Fragments:

DOTA binding Fab: The DOTA binding Fab used in the Examples are derived from the 2D12 antibody (Corneillie et al. J. Am. Chem. Soc. 125:15039-15048 (2003)). In one example (CD38-DOTA (humanized fab)-p53) introducing the substitutions identified in the affinity maturation proceed disclosed in WO10099536, and humanization thereof was incorporated. The DOTA binding Fab consists of a light chain (DOTA-FAB-LC) with the amino acid sequence disclosed as amino acids no: 133-347 of SEQ ID NO: 10; and a heavy chain (DOTA-FAB-HC) with the amino acid sequence disclosed as amino acids no: 1-222 of SEQ ID NO: 11.

DOTAM binding Fab: The DOTAM binding Fab used in the Examples are derived from the DOTAM binding antibody disclosed in WO2019/201959. The DOTAM binding Fab consists of a light chain (DOTAM-FAB-LC) with the amino acid sequence disclosed as amino acids no: 146-364 of SEQ ID NO: 16 and a heavy chain (DOTAM-FAB-HC) with the amino acid sequence disclosed as amino acids 1-224 of SEQ ID NO: 17.

SPR analysis was performed on a Biocore 8K+, using a CFJB0944 CM5 sensor chip for immobilization and a SPR running buffer 1×HBS-EP+pH 7.4 (Cytiva, cat no. BR100669) according to the manufacturer's instructions.

### EXAMPLES

### Example 1. Production of Molecules of the Invention Comprising HER2 Binding Site or CD38 Binding Site

This example demonstrates the production of exemplary bispecific compounds with a first binding domain that binds a cellular target (e.g. a cell surface target), a second binding domain that binds a payload, and a tetramerization domain. Specifically, this example describes the production of 2 exemplary bispecific antibody-based compounds CD38-DOTA (Fab)-p53 and HER2-DOTA (Fab)-p53, comprising a VHH linked by a G4S linker to a FAB DOTA binder with a C-terminal p53 tetramerization domain and a His-tag.

In order to do that, a HEK suspension cell line was transformed with an expression cassette encoding a first polypeptide (SEQ ID NO: 12) consisting of a VHH against CD38, a (G4S)₄, and DOTA-FAB_LC and a second polypeptide (SEQ ID NO: 11) consisting of DOTA-FAB_HC with C-terminal p53 and a His-tag. (SEQ ID NO:11). This molecule was named CD38-DOTA (Fab)-p53.

HEK transformants were cultured in expression medium and the product was captured from the media after centrifugation, using protein L resin.

Same procedure was conducted to make a molecule—HER2-DOTA (Fab)-p53—of the invention with a HER2 binding site with a first polypeptide (SEQ ID NO: 10) comprising a VHH against HER2, a (G4S)₄ and DOTA-FAB_LC and same second polypeptide (SEQ ID NO: 11) as used in CD38-DOTA (Fab)-p53.

### Example 2. Analysis of Exemplary Bispecific Compounds

The two compounds prepared in example 1 were analysed by HPLC-SEC, and the chromatograms are shown in FIGS. 2A and 2B.

The HPLC-SEC analysis showed only one peak, appearing nicely symmetrical without shoulders for both HER2-DOTA (Fab)-p53 and CD38-DOTA (Fab)-p53 respectively, indicating that the products were uniform and no compounds with different molecular weight or aggregates could be seen.

### Example 3. Production of Additional Molecules of the Invention

The procedure described in example 1 was used to make additional molecules, as listed in Table 1 below. Please note that the two molecules prepared in Example 1 are included in the table as molecule no. 1 and 2, and CD38-DOTA (fab)-dp53 from example 4 is included as molecule no. 15. Linkers and His-tags are not mentioned in the table, but the full sequences can be seen in the Sequence listing.

TABLE 1
produced molecules of the invention

| Molecule | | | first chain | Sequence |
|---|---|---|---|---|
| Number | Reference | Description | second chain | Sequence |
| 1 | YM00016 | CD38-DOTA(Fab)-p53 | CD38-VHH/LC_DOTA | SEQ ID NO: 12 |
| | | | HC_DOTA/p53 | SEQ ID NO: 11 |
| 2 | YM00018 | HER2-DOTA(Fab)-p53 | HER2-VHH/LC_DOTA | SEQ ID NO: 10 |
| | | | HC_DOTA/p53 | SEQ ID NO: 11 |
| 3 | YM00004 | CD38 (bivalent)- | CD38-VHH/LC-DOTA | SEQ ID NO: 12 |
| | | DOTA(Fab)-p53 | CD38-VHH/HC- | SEQ ID NO: 14 |
| | | | DOTA/p53 | |
| 4 | YM00005 | CD38 (Bivalent) - | CD38-VHH/LC-DOTA | SEQ ID NO: 12 |
| | | DOTA(Fab)-dp53 | CD38-VHH/HC-DOTA | SEQ ID NO: 15 |
| 5 | YM00006 | CD38-DOTAM(Fab)-p53 | CD38-VHH/LC- | SEQ ID NO: 16 |
| | | | DOTAM | |
| | | | HC-DOTAM/p53 | SEQ ID NO: 17 |
| 6 | YM00007 | CD38-DOTAM(fab)- | CD38-VHH/LC- | SEQ ID NO: 16 |
| | | dp53 | DOTAM | |
| | | | HC-DOTAM | SEQ ID NO: 18 |
| 7 | YM00008 | BAFF-DOTA(fab)-p53 | BAFF-VHH/LC-DOTA | SEQ ID NO: 19 |
| | | | HC-DOTA/p53 | SEQ ID NO: 27 |
| 8 | YM00009 | CD33-DOTA(fab)-p53 | CD33-VHH/LC-DOTA | SEQ ID NO: 20 |
| | | | HC-DOTA/p53 | SEQ ID NO: 27 |
| 9 | YM00010 | EGFR-DOTA(fab)-p53 | EGFR-VHH/LC-DOTA | SEQ ID NO: 21 |
| | | | HC-DOTA/p53 | SEQ ID NO: 27 |
| 10 | YM00011 | CEA-DOTA(fab)-p53 | CEA-VHH/LC-DOTA | SEQ ID NO: 22 |
| | | | HC-DOTA/p53 | SEQ ID NO: 27 |
| 11 | YM00012 | CD276-DOTA(fab)-p53 | CD276-VHH/LC- | SEQ ID NO: 26 |
| | | | DOTA | |
| | | | HC-DOTA/p53 | SEQ ID NO: 27 |
| 12 | YM00013 | CD38- DOTA (fab)- | LC-DOTA/CD38-VHH | SEQ ID NO: 23 |
| | | p53_C-Term-LC | HC-DOTA/p53 | SEQ ID NO: 27 |
| 13 | YM00014 | CD38- DOTA (fab)- | LC-DOTA | SEQ ID NO: 24 |
| | | p53_C-Term-HC | HC-DOTA/CD38- | SEQ ID NO: 25 |
| | | | VHH/p53 | |
| 14 | YM00015 | CD38(Bivalent)- | LC-DOTA/CD38-VHH | SEQ ID NO: 23 |
| | | DOTA(fab)-p53_C-Term | HC-DOTA/CD38- | SEQ ID NO: 25 |
| | | | VHH/p53 | |
| 15 | YM00017 | CD38-DOTA(fab)-dp53 | CD38-VHH/LC-DOTA | SEQ ID NO: 12 |
| | | | HC-DOTA/DP53 | SEQ ID NO: 13 |
| 16 | YM00032 | CD38-DOTA(humanized | CD38-VHH/hLC-DOTA | SEQ ID NO: 28 |
| | | fab)-p53 | hHC-DOTA | SEQ ID NO: 29 |
| 17 | YM00053 | HER2/CD276_23F11- | HER2-VHH/hLC | SEQ ID NO: 30 |
| | | DOTA(humanized fab)- | DOTA | |
| | | p53 | CD276_23F11-hHC- | SEQ ID NO: 31 |
| | | | DOTA | |
| 18 | YM00052 | HER2/CD276 23A04- | HER2-VHH/hLC | SEQ ID NO: 30 |
| | | DOTA(humanized fab)- | DOTA | |
| | | p53 | CD276_23A04-hHC- | SEQ ID NO: 32 |
| | | | DOTA | |

The compounds prepared in this example were analyzed by HPLC-SEC, and as examples of the resulting chromatograms, chromatograms for YPRIT_CD38-DOTA (fab)-p53_C-Term-LC (Molecule 12) and CD33-DOTA (fab)-p53 (molecule 8) are shown in FIGS. 3A and 3B.

The HPLC-SEC analysis showed only one peak, appearing nicely symmetrical without shoulders for all analysed molecules, indicating that the products were uniform and no compounds with different molecular weight or aggregation could be seen.

### Example 4. Production and Analysis of Exemplary Bispecific Compound without Tetramerization Domain

This example demonstrates the production of exemplary bispecific compound with a first binding domain that binds a cellular target (e.g. a cell surface target), and a second binding domain that binds a payload. Specifically, this example describes the production of an exemplary bispecific antibody-based compound CD38-DOTA (fab)-dp53 comprising a VHH linked by a G4S linker to a FAB DOTA (molecule 15 of example 3) and a His-tag.

In order to do that, a HEK suspension cell line was transformed with an expression cassette encoding a first polypeptide (SEQ ID NO: 12) consisting of a VHH against CD38, a (G4S)₄, DOTA-FAB-LC; and a second polypeptide (SEQ ID NO: 13) consisting of DOTA-FAB-HC and a His-tag. This molecule was named CD38-DOTA (fab)-dp53.

HEK transformants were cultured in expression medium and the product was captured from the media after centrifugation, using protein L resin.

The compound was analysed by HPLC-SEC, and the chromatogram is shown in FIG. 4.

The HPLC-SEC analysis showed only 1 peak, appearing nicely symmetrical without shoulders indicating that the product was uniform and no compounds with different molecular weight or aggregates could be seen. Also observed is a later retention time that the corresponding molecule with p53 in accordance with the monomeric and not tetrameric size of the product.

### Example 5. Colloidal Stability I (DLS)

This example describes the colloidal stability of exemplary bispecific compounds HER2-DOTA (Fab)-p53 and CD38-DOTA (Fab)-p53 (molecules 1 and 2 of example 3).

Dynamic light scattering (DLS) was used to determine the hydrodynamic diameter of CD38-YPRIT and HER2-YPRIT.

Results are shown in FIG. 5 and in table 2 below.

TABLE 2
DLS Results:

| | Intensity Distribution | |
|---|---|---|
| Annotations | Peak 1 (nm) | PDI 1 |

| Number | Sample ID | Ø | σ | Ø | σ |
|---|---|---|---|---|---|
| 1 | HER2- | 7.37 | 0.03 | 0.03 | 0.01 |
| | DOTA(Fab)- | | | | |
| | p53 | | | | |
| 2 | CD38- | 7.69 | 0.04 | 0.05 | 0.01 |
| | DOTA(Fab)- | | | | |
| | p53 | | | | |

For HER2-DOTA (Fab)-p53 a radius of 7.37 nm was detected. The polydispersity was very low (PDI=0.03) indicating a highly monodisperse sample.

For CD38-DOTA (Fab)-p53 a radius of 7.69 nm was detected. The polydispersity was very low (PDI=0.05) indicating a highly monodisperse sample.

Thus, both samples showed high colloidal stability.

### Example 6. Colloidal Stability 1b (DLS)

Colloid stability of the following 6 molecules prepared in example 3 were described using dynamic light scattering (DLS) to determine the hydrodynamic diameter:
CD38 (Bivalent)-DOTA (fab)-dp53-Molecule 4 of Table 1
CD38-DOTAM (fab)-p53-Molecule 5 of Table 1
CD38-DOTAM (fab)-dp53-Molecule 6 of Table 1
CD38-DOTA (fab)-p53_C-Term-LC-Molecule 12 of Table 1
CD38-DOTA (fab)-p53_C-Term-HC-Molecule 13 of Table 1
CD38 (Bivalent)-DOTA (fab)-p53_C-Term-Molecule 14 of Table 1.

TABLE 3
DLS Results:

| Annotations | | Cumulant Fit | | | |
|---|---|---|---|---|---|
| Molecule | | Cumulant Radius (nm) | | Cumulant PDI | |
| Number | Target name | Ø | σ | Ø | σ |
| 4 | CD38 (bivalent)- | 4.23 | 0.03 | 0.07 | 0.01 |
| | DOTA(Fab)-dp53 | | | | |
| 5 | CD38- | 7.32 | 0.01 | 0.00 | 0.00 |
| | DOTAM(fab)-p53 | | | | |
| 6 | CD38- | 3.66 | 0.02 | 0.03 | 0.01 |
| | DOTAM(fab)- | | | | |
| | dp53 | | | | |
| 12 | CD38-DOTA(fab)- | 6.78 | 0.07 | 0.02 | 0.01 |
| | p53_C-Term-LC | | | | |
| 13 | CD38-DOTA(fab)- | 7.33 | 0.01 | 0.03 | 0.03 |
| | p53_C-Term-HC | | | | |
| 14 | CD38(Bivalent)- | 7.82 | 0.13 | 0.04 | 0.04 |
| | DOTA(fab)-p53_C- | | | | |
| | Term | | | | |

A hydrodynamic radius between 4.23 to 7.82 nm was observed for the variants. The PDI was <0.1 (0.00 to 0.07) for all six samples indicating highly monodisperse sample quality.

Thus, all samples showed high colloidal stability and homogenous sample quality. Also evident is the smaller size of constructs without the p53 domain (molecule 4 and 6).

### Example 7. Colloidal Stability II (Nano DSF)

This example describes the thermal stability of exemplary bispecific compounds HER2-DOTA (Fab)-p53 and CD38-DOTA (Fab)-p53 (molecules 2 and 1 of Table 1).

Nano Differential Scanning Flourimetry (DSF) was used to determine unfolding transitions and sample aggregation.

Results are shown in FIG. 6 and table 4 below.

TABLE 4
nanoDSF Results:

| | | IP | IP | | |
|---|---|---|---|---|---|
| Annotations | | #1 | #2 | ON | Turbidity [symbol] |
| Number | Sample ID | (° C.) | (° C.) | (° C.) | ON (° C.) |
| 1 | HER2-DOTA(Fab)- | 63.55 | 76.09 | 55.34 | 61.32 |
| | p53 | | | | |
| 2 | CD38-DOTA(Fab)- | 62.72 | 75.37 | 56.05 | 53.57 |
| | p53 | | | | |

As can be seen from the table 4 above and FIG. 6 for HER2-DOTA (Fab)-p53 two unfolding transitions were observed at 63.55° C. and 76.09° C. Protein unfolding (Ratio T_{ON}) started at 55.34° C. Microscopic sample aggregation was observed from 61.32° C. (Turbidity T_{ON}). Scattering amplitude was high and signal/noise ratio was very good.

For CD38-DOTA (Fab)-p53 two unfolding transitions were observed at 62.72° C. and at 75.37° C. Protein unfolding (Ratio T_{ON}) started at 56.05° C. Microscopic sample aggregation was observed from 53.57° C. (Turbidity T_{ON}). Scattering amplitude was high and signal/noise ratio was very good.

Thus, both proteins showed unfolding profiles indicating good colloidal stability.

### Example 8. Colloidal Stability IIb (Nano DSF)

Colloid stability of the following 6 molecules prepared in example 3 were described using Nano Differential Scanning Flourimetry (DSF) to determine unfolding transitions and sample aggregation:
CD38 (Bivalent)-DOTA (fab)-dp53-molecule 4 of Table 1
CD38-DOTAM (fab)-p53-molecule 5 of table 1
CD38-DOTAM (fab)-dp53-molecule 6 of table 1
CD38-DOTA (fab)-p53_C-Term-LC-molecule 12 of table 1
CD38-DOTA (fab)-p53_C-Term-HC-molecule 13 of table 1
CD38 (Bivalent)-DOTA (fab)-p53_C-Term molecule 14 of table 1.

TABLE 5
nano DSF results:

| | | Annotations | | | | | |
|---|---|---|---|---|---|---|---|
| | | 350 nm [symbol] | | | | Turbidity [symbol] | |
| Molecule | | IP #1 (° C.) | | IP #2 (° C.) | | ON (° C.) | |
| Number | Target name | ø | σ | ø | σ | ø | σ |
| 4 | CD38 (Bivalent) - | 69.86 | 0.05 | | | 62.94 | 0.77 |
| | DOTA(fab)-dp53 | | | | | | |
| 5 | CD38-DOTAM(fab)-p53 | 69.16 | 0.09 | | | 68.18 | 0.41 |
| 6 | CD38-DOTAM(fab)-dp53 | 69.11 | 0.05 | | | 55.61 | 0.51 |
| 12 | CD38-DOTA(fab)-p53_C- | 68.38 | 0.08 | | | 64.60 | 3.32 |
| | Term-LC | | | | | | |
| 13 | CD38-DOTA(fab)-p53_C- | 68.20 | 0.02 | 75.99 | 0.00 | 66.84 | 0.05 |
| | Term-HC | | | | | | |
| 14 | CD38(Bivalent)- | 67.88 | 0.23 | | | 53.48 | 10.06 |
| | DOTA(fab)-p53_C-Term | | | | | | |

As can be seen from the table above one main unfolding transition was observed between ˜68° C. (IP #1) to 69° C. For CD38-DOTA (fab)-p53_C-Term-HC a second unfolding transition was observed.

Macroscopic sample aggregation was observed from 53° C. to 68° C. (Turbidity TON). The scattering amplitude was high and signal/noise ratio was very good.

Thus, the proteins showed unfolding profiles indicating high colloidal and thermal stability.

### Example 9. SPR Data: DOTA Binding In Vitro

This example documents the binding characteristics of the exemplary bispecific compounds HER2-DOTA (Fab)-p53 and C CD38-DOTA (Fab)-p53 to Lu-DOTA.

Surface Plasmon Resonance was used to determine association, dissociation and equilibrium constants.

TABLE 6
SPR

| | | | Replicate | ka | | | Chi2/ |
|---|---|---|---|---|---|---|---|
| Sample | Ligand | Analyte | M140-LU | (1/Ms) | kd (1/s) | KD (M) | Rmax |
| HER2- | DOTA- | Sample | 1 | 6.15E+03 | 1.03E−04 | 1.67E−08 | 0% |
| DOTA(Fab)- | Lu | | 2 | 8.32E+03 | 1.64E−04 | 1.97E−08 | 1% |
| p53 | | | Mean | 7.24E+03 | 1.34E−04 | 1.82E−08 | |
| | | | SD | 1.54E+03 | 4.31E−05 | 2.09E−09 | |
| | | | % CV | 21% | 32% | 11% | |
| CD38- | DOTA- | Sample | 1 | 7.13E+03 | 1.89E−04 | 2.65E−08 | 1% |
| DOTA(Fab)- | Lu | | 2 | 7.20E+03 | 2.48E−04 | 3.44E−08 | 1% |
| p53 | | | Mean | 7.16E+03 | 2.18E−04 | 3.05E−08 | |
| | | | SD | 5.52E+01 | 4.18E−05 | 5.59E−09 | |
| | | | % CV | 1% | 19% | 18% | |

It demonstrated that exemplary HER2-DOTA (Fab)-p53 and CD38-DOTA (Fab)-p53 effectively bind in vitro to Lu-DOTA.

### Example 10. SPR Data, Tumor Antigen Binding In Vitro

This example documents the binding characteristics of the exemplary bispecific compounds HER2-DOTA (Fab)-p53 and CD38-DOTA (Fab)-p53 to bio-huHER2/Erb2 and bio-huCD38 respectively. Further it documents the binding characteristics of exemplary bispecific compound CD38-DOTA (fab)-dp53.

Surface Plasmon Resonance was used to determine association, dissociation and equilibrium constants.

TABLE 7
SPR

| | Immobilized | Concentration | ka | kd | KD |
|---|---|---|---|---|---|
| Captured antibody | antigen | (nM) | (1/Ms) | (1/s) | (M) |
| HER2-DOTA(Fab)- | Bio-huHER2/ | 25.0-1.6 | 3.83E+05 | 2.25E−05 | 5.88E−11 |
| p53 | Erb2 | | | | |
| CD38-DOTA(Fab)- | Bio-huCD38 | 25.0-1.6 | 5.50E+05 | 1.41E−05 | 2.57E−11 |
| p53 | | | | | |
| CD38-DOTA(fab)- | | | 4.70E+05 | 1.48E−04 | 3.16E−10 |
| dp53 | | | | | |

It shows that exemplary HER2-DOTA (Fab)-p53 and CD38-DOTA (Fab)-p53 effectively bind in vitro to their respective tumor targets. Obtained values for CD38-DOTA (fab)-dp53 suggest that the addition of the p53 potentiates binding by avidity to bio-huCD38.

### Example 11. SPR Data, Tumor Antigen Binding In Vitro

SPR analysis was performed essentially as outlined in Example 10.

In the first part of this example the antibody was immobilized on the chip and the antigen dissolved in the SPR running buffer flowed over the chip. This gave the measurements shown in Table 8.

TABLE 8
SPR: Antibody as ligand set-up

| Captured molecule | | Concentration | ka | kd | KD |
|---|---|---|---|---|---|
| (Molecule number) | Analyte | (nM) | (1/Ms) | (1/s) | (M) |
| CD38 (bivalent)- | bio-huCD38 | 200-6.25 | 4.32E+04 | 1.20E−04 | 2.77E−09 |
| DOTA(Fab)-p53 (3) | | | | | |
| CD38 (Bivalent) - | bio-huCD38 | 200-6.25 | 4.47E+04 | 1.12E−03 | 2.51E−08 |
| DOTA(fab)-dp53 (4) | | | | | |
| CD38-DOTAM(fab)- | bio-huCD38 | 200-6.25 | 3.76E+04 | 7.86E−05 | 2.09E−09 |
| p53 (5) | | | | | |
| CD38-DOTAM(fab)- | bio-huCD38 | 200-6.25 | 1.68E+04 | 2.14E−04 | 1.27E−08 |
| dp53 (6) | | | | | |
| CD33-DOTA(fab)-p53 | bio-huCD33 | 1500-296.3 | 1.39E+03 | 5.55E−03 | 3.99E−06 |
| (8) | | | | | |
| EGFR-DOTA(fab)-p53 | bio-huEGFR | 1500-296.3 | N/D | N/D | N/D |
| (9) | | | | | |
| CEA-DOTA(fab)-p53 | bio-huCEACAM-5 | 200-6.25 | 1.74E+04 | 1.20E−05 | 6.92E−10 |
| (10) | | | | | |
| CD276-DOTA(fab)-p53 | bio-huCD276 | 400-12.5 | 1.14E+05 | 4.56E−03 | 4.01E−08 |
| (11) | | | | | |
| CD38-DOTA(fab)- | bio-huCD38 | 200-6.25 | 8.07E+04 | 1.03E−04 | 1.28E−09 |
| p53_C-Term-LC (12) | | | | | |
| CD38-DOTA(fab)- | bio-huCD38 | 200-6.25 | 5.70E+04 | 7.59E−05 | 1.33E−09 |
| p53_C-Term-HC (13) | | | | | |
| CD38(Bivalent)- | bio-huCD38 | 200-6.25 | 3.38E+04 | 6.41E−05 | 1.89E−09 |
| DOTA(fab)-p53_C- | | | | | |
| Term (14) | | | | | |

In the second part of this example, antigen was immobilized on the chip and antibody dissolved in the SPR running buffer flowed over the chip. This gave the measurements shown in table 9.

TABLE 9
SPR: Antigen as ligand set-up

| Antibody (molecule | Immobilized | Concentration | ka | kd | KD |
|---|---|---|---|---|---|
| number) | antigen | (nM) | (1/Ms) | (1/s) | (M) |
| CD38 (bivalent)- | bio-huCD38 | 25-1.6(*) | 1.21E+05 | <1.00E−07 * | <8.26E−13 ** |
| DOTA(Fab)-p53 (3) | | | | | |
| CD38 (Bivalent) - | bio-huCD38 | 25-1.6 | 3.24E+05 | <1.00E−07 * | <3.09E−13 |
| DOTA(fab)-dp53 (4) | | | | | |
| CD38-DOTAM(fab)- | bio-huCD38 | 25-1.6 | 1.74E+04 | 1.63E−05 | 9.36E−10 |
| p53 (5) | | | | | |
| CD38-DOTAM(fab)- | bio-huCD38 | 25-1.6 | 1.43E+06 | 1.89E−04 | 1.32E−10 |
| dp53 (6) | | | | | |
| CD33-DOTA(fab)-p53 | bio-huCD33 | 200-12.5 | 2.11E+03 | 1.42E−06 | 6.75E−10 |
| (8) | | | | | |
| EGFR-DOTA(fab)-p53 | bio-huEGFR | 25-1.6 | 4.64E+05 | 9.85E−04 | 2.12E−9 |
| (9) | | | | | |
| CEA-DOTA(fab)-p53 | bio-huCEACAM-5 | 25-1.6 | 4.90E+04 | 4.62E−06 | 9.44E−11 |
| (10) | | | | | |
| CD276-DOTA(fab)-p53 | bio-huCD276 | 25-1.6 | 9.05E+03 | 2.26E−05 | 2.50E−09 |
| (11) | | | | | |
| CD38-DOTA(fab)- | bio-huCD38 | 100-6.25 | 4.15E+04 | 1.30E−05 | 3.13E−10 |
| p53_C-Term-LC (12) | | | | | |
| CD38-DOTA(fab)- | bio-huCD38 | 50-3.1 | 9.33E+04 | 1.52E−05 | 1.63E−10 |
| p53_C-Term-HC (13) | | | | | |
| CD38(Bivalent)- | bio-huCD38 | 25-1.6 | 1.87E+05 | <1.00E−07 * | <5.35E−13 ** |
| DOTA(fab)-p53_C- | | | | | |
| Term (14) | | | | | |

* kd value could not be determined due to limitations of the instrument, a KD value was estimated based on the obtained ka and the limit kd level of detection of the instrument (1.00E−07).
** Value to be regarded as indicative due to the reason described in *.

From tables 8 and 9 it was concluded that the exemplary molecules effectively bound in vitro to their respective tumor targets, which confirmed that molecules against different tumor-targets could be generated. Further, it showed that molecules with different valency could be generated and that both N and C-terminal VHH attachment could be generated.

### Example 12. cIEF Heterogeneity

Capillary isoelectric focusing was applied for antibody charge heterogeneity analysis, where molecules were separated based on isoelectric point (pI).

The analysis was conducted for the exemplary bispecific compounds CD38-DOTA (Fab)-p53 and CD38-DOTA (fab)-dp53, molecule 1 and 15 of table 1.

As can be seen from FIG. 7. CD38-DOTA (Fab)-p53 has a very heterogenious cIEF profile and a distinct main peak is difficult to annotate.

As can be seen from FIG. 8 CD38-DOTA (Fab)-dp53 has a cIEF profile with a main peak at 9.45 and two minor acidic peaks at 9.25 and 8.99.

These data show that deleting the tetramerization domain results in a much more homogenous charge population compared to the YPRIT having a C-terminal P53 domain.

### Reference Example 1: CD38 SADA

This example is included as reference and is originally disclosed in example 4 of the application PCT/DK2022/050280.

In this example variants of bispecific antibodies capable of binding CD38 and DOTA were generated.

Each variant consisted of anti-CD38 scFv, an anti-DOTA scFv and a SADA domain and differs only in the number of interchain disulfide bonds within the scFvs. More details can be found in PCT/DK2022/050280. The constructs in table 10 were generated.

TABLE 10
Generated constructs

| | antiCD38 scFv | Anti-DOTA | SADA |
|---|---|---|---|
| YMS9a | Containing one VH- | Containing one VH-VL | P53 domain (SEQ ID NO: 1, |
| | VL disulfide bond | disulfide bond | amino acids 6 to 36) |
| YMS9c | Containing one VH- | Without VH-VL | P53 domain (SEQ ID NO: 1, |
| | VL disulfide bond | disulfide bond | amino acids 6 to 36) |
| YMS9d | Without VH-VL | Without VH-VL | P53 domain (SEQ ID NO: 1, |
| | disulfide bond | disulfide bond | amino acids 6 to 36) |

The constructs were analyzed on non-reducing SDS-PAGE, See FIG. 9.

The results show that YMS9a and YMS9c contained significant amounts of multimers, whereas the amount of multimers were significantly diminished or absent in YMS9d.

The results also showed that YMS9a and YMS9c gave rise to some heterogeneity in the monomer band. The heterogeneity disappeared under reducing conditions.

### Example 13 Tetramerization

Sample molecules were dissolved in PBS at different concentrations and allowed to reach equilibrium by incubating for 180 min at 37° C. The samples were then analyzed for their monomer and tetramer content using a Refeyn Two Mass Photometer, with the results being expressed as the number of monomer molecules that are either in monomeric or tetrameric state calculated as a percentage of the total number of available monomers.

The molecules used for this example were molecules 1 and 2 from table 1 and the CD38-SADA molecule of example 12 named YMS9d.

Results can be seen in FIG. 10.

The results showed that the molecules of the invention are capable of tetramerize, and monomerize dependent on concentration and that the distribution between monomers and tetramers is similar to the distribution of a corresponding SADA molecule.

### Example 14 Analysis of Mouse PK Samples

ELISA was applied for analysis of mouse PK samples to evaluate the level of CD38-molecules in BALB/C mouse plasma.

In order to determine the clearing of molecules of the invention, sample molecules of the invention were administered to BALB/C mice, blood samples were collected at different times after the administration and the concentration of the sample molecules determined using ELISA.

TABLE 11
CD38 variants tested:

| | | Molecule | Protein | | |
|---|---|---|---|---|---|
| | | number cf. | conc | Amount | Volume |
| Group | Name | table 1 | (mg/ml) | (mg) | (mL) |
| 1 | CD38(Bivalent)- | 14 | 2.49 | 0.249 | 0.1 |
| | DOTA(fab)- | | | | |
| | p53_C-Term | | | | |
| 2 | CD38-DOTA(fab)- | 13 | 5.04 | 0.504 | 0.1 |
| | p53_C-Term-HC | | | | |
| 3 | CD38-DOTA(fab)- | 12 | 1.77 | 0.177 | 0.1 |
| | p53_C-Term-LC | | | | |
| 4 | CD38 (Bivalent) - | 4 | 5.81 | 0.581 | 0.1 |
| | DOTA(fab)-dp53 | | | | |
| 5 | CD38-DOTAM(fab)- | 3 | 5.09 | 0.509 | 0.1 |
| | p53 | | | | |

The ELISA assay was initially tested and optimized with CD38-DOTA (humanized fab)-p53 and showed acceptable precision and accuracy in both assay buffer and 1% BALB/C plasma matrix.

Each of the five molecules were tested in 1% BALB/C plasma matrix in three separate experiments for each variant. The results showed acceptable precision and accuracy for all five variants. Performance of the five variants was comparable to performance of the CD38-DOTA (humanized fab)-p53 variant.

The pk curves are shown in FIGS. 11A-11E.

The results showed fast clearing of the molecules at a rate similar to the clearing rate observed for CD38-SADA (Data not shown). The fast PK of the tested molecules confirms that the molecules of the invention are suitable for 2-step PRIT essentially using the 2-step PRIT method disclosed in WO2018204873, without the need for administrating a clearing agent between the tumor binding bispecific molecule and the administration of the chelator binding the radionuclide.

### Example 15. EC50 Determination of YPRIT Molecules by Binding Fluorescence-Activated Cell Sorting (FACS)

QC of Daudi, THP-1, A-431, HEK293T, MKN-45 and Jurkat E6-1 cell lines for CD38, CD33, EGFR, CEA and CD276 expression prior to use in EC50 determination by FACS, using reference antibodies were performed.

Subsequently the EC50 of antibodies samples was determined by titration binding FACS on Daudi cells (for CD38 binding evaluation), on THP-1 cells (for CD33 binding evaluation), on A-431 cells (for EGFR binding evaluation), on MKN-45 cells (for CEACAMS binding evaluation) and on HEK293T (for CD276 binding evaluation). Jurkat E6-1 or HEK293T cell lines were used as negative control, as applicable.

As can be seen from FIGS. 12A-12D, EGFR-DOTA (fab)-p53 and CD276-DOTA (fab)-p53 showed specific binding, with no binding to the control cell line Jurkat E6-1. CEA-DOTA (fab)-p53 showed specific binding to MKN-45, with no binding to the control cell line HEK293T. CD33-DOTA (fab)-p53 showed background binding to the Jurkat E6-1 cell line and the HEK293T cell line.

All CD38 YPRIT molecules showed binding with full titration in Daudi cells in the range of concentrations tested, and with similar determined EC50 For CD38 no background binding was observed on HEK293T cells. FIGS. 12E-12H show the titration curves for CD38-DOTA (fab)-p53, CD38-DOTA (fab)-dp53, CD38 (bivalent)-DOTA (Fab)-p53 and CD38 (bivalent)-DOTA (Fab)-dp53 respectively.

The trivalent molecules HER2/CD276_23F11-DOTA (humanized fab)-p53 and HER2/CD276_23A04-DOTA (humanized fab)-p53 showed specific binding, with no binding to the control cell line Jurkat E6-1 as can be seen from FIGS. 12I-12J.

Obtained EC50 values for the YPRIT molecules can be seen from table 12.

TABLE 12
EC50 Values for YPRIT molecules.

| | Molecule | EC50 |
|---|---|---|
| YPRIT sample | number | (nM) |
| CD38 (bivalent)- DOTA(Fab)-p53 | 3 | 3.45 |
| CD38 (Bivalent) - DOTA(fab)-dp53 | 4 | 0.32 |
| CD38-DOTAM(fab)-p53 | 5 | 0.59 |
| CD38-DOTAM(fab)-dp53 | 6 | 0.51 |
| CD38-DOTA(fab)-p53_C-Term-LC | 12 | 3.75 |
| CD38-DOTA(fab)-p53_C-Term-HC | 13 | 2.36 |
| CD38(Bivalent)-DOTA(fab)-p53_C-Term | 14 | 3.24 |
| CD38-DOTA(Fab)-p53 | 1 | 0.65 |
| CD38-DOTA(fab)-dp53 | 15 | 1.07 |
| EGFR-DOTA(fab)-p53 | 9 | 51.48 |
| CEA-DOTA(fab)-p53 | 10 | 4.81 |
| CD33-DOTA(fab)-p53 | 8 | N.D |
| CD276-DOTA(fab)-p53 | 11 | 2.14 |
| HER2/CD276_23F11-DOTA(humanized fab)-p53 | 17 | 12.36 |
| HER2/CD276_23A04-DOTA(humanized fab)-p53 | 18 | 90.16 |

For CD33-DOTA (fab)-p53 we do see binding to cells above the background of HEK293T and Jurkat cells but the concentration range does not allow for determination of an EC50. These data show that we can produce tumor binding YPRITS using VHH sequences targeting a broad panel of sequences. Our data also show that some VHH might provide unspecific binding to control cells. As the VHH sequences in these examples are selected from published sources it suggests that a selection from a panel of candidate VHH sequences would be beneficial when engineering a YPRIT with optimal clinical profile.

### Example 16 In Vivo Distribution

For this example, BRGSF mice with a CD38 positive tumor implanted were provided.

Sample molecules of the invention were administered to the mice. Following a delay time after administration of the molecules a single administration of ¹⁷⁷Lu-DOTA was given to the mice. The delay time was 24 hours for CD38-DOTA (humanized fab)-p53, CD38 (bivalent)-DOTA (Fab)-p53 and CD38 (Bivalent)-DOTA (Fab)-dp53, 48 hours for CD38-DOTA (fab)-p53_C-Term-HC and CD38-DOTA (Fab)-p53 and 72 hours for CD38-DOTA (fab)-p53_C-Term-LCand CD38 (Bivalent)-DOTA (fab)-p53_C-Term.

The mice were scanned in a SPECT scanner, 2, 24 and 48 hours after 177Lu-DOTA administration and the tissue distribution of ¹⁷⁷Lu was determined by image analysis of the scanned images.

The results showed that the tested molecules of the invention bound well to the tumor antigen in vivo. Further, tumor uptake was higher or similar to reference CD38-SADA molecule (not shown).

FIG. 13A-13B shows the in vivo distribution assessed using image analysis of the scanned images for CD38-DOTA (Fab)-p53 (molecule 1) and CD38-DOTA (humanized fab)-p53 (molecule 16).

### Example 17

In a subsequent study the groups of animal receiving the following test compounds: CD38 (Bivalent)-DOTA (fab)-p53_C-Term, CD38-DOTA (fab)-p53_C-Term-HC, CD38-DOTA (fab)-p53_C-Term-LC, CD38 (Bivalent)-DOTA (fab)-dp53 and CD38 (bivalent)-DOTA (Fab)-p53. Animals were euthanized after the SPECT scan at 48 h post 177Lu-DOTA administration and the following samples were collected: blood, bone, kidneys, large intestine, liver, muscle, small intestine, spleen, stomach, tail and tumor. They were transferred to dry plastic tubes for radioactivity content measurement. FIGS. 14A-14E show the tissue biodistribution for CD38 (bivalent)-DOTA (Fab)-p53, CD38 (Bivalent)-DOTA (fab)-dp53, CD38-DOTA (fab)-p53_C-Term-LC, CD38-DOTA (fab)-p53_C-Term-HC and CD38 (Bivalent)-DOTA (fab)-p53_C-Term respectively.

The results showed a higher tumor-to-kidney ratio for CD38 (Bivalent)-DOTA (fab)-dp53 and CD38 (Bivalent)-DOTA (fab)-p53, in particular CD38 (Bivalent)-DOTA (fab)-dp53.

Further, the bivalent CD38-YPRITS provided for a better tumor to kidney ratio than the monovalent CD38-YPRITS with C-terminal VHH geometry.

### Example 18: Binding to Pb-TCMC-PEG4-Biotin In Vitro

This example documents the binding characteristics of YPRIT compounds to Pb-TCMC-PEG4-Biotin, which is a biotinylated variant of Pb-DOTAM.

Plates with wells were coated with SADA/YPRIT (1 μg/mL, 100 μL per well) overnight at 4 C, after which the plates were washed 2× with plate washer, blocked with PBST+1% BSA for 1 h at room temperature and washed 2×.

Diluted Pb-TCMC-PEG4-Biotin was added to the coated wells and incubated at RT for 90 min on a plate shaker (400 rpm), 100 μl each well and the plates were washed 5×. Streptavidin-HRP (1:5,000, 100 μL per well) was added and incubated at room temperature for 30 min on a plate shaker (400 rpm), 100 μL each well and the plates were washed 5×. TMB substrate was added and incubated at RT for 15 min. 100 μL each well. Stop solution was added, 100 μl each well and the plates were read at 450 nm with Synergy H1.

CD38-SADA (YMS9a, see reference example 1) was used as negative control, as it only binds Lu-DOTA and a variant of YMS9a that contains an anti-Pb-DOTAM scFv instead of the anti-DOTA scFv (Sequence not shown) was used as positive control, because it uses the same anti-Pb-DOTAM sequence as the test YPRITS.

From FIG. 15, it can be seen that the positive control, YPRIT-Pb-DOTAM-p53 and YPRIT-Pb-DOTAM-dp53 all binds Pb-TCMC-PEG4-Biotin in a concentration dependent manner. The EC50 of binding for the YPRIT-Pb-DOTAM-p53 and YPRIT-Pb-DOTAM-dp53 are 1.1 and 1.2 ng/ml respectively, showing that affinities for Pb-TCMC are in the same range.

1. A compound comprising:
a. A first antigen binding site capable of binding a tumor antigen; and
b. A second antigen binding site capable of binding a chelator;
wherein the first antigen binding site is selected among Fab fragments and VHH binding fragments; the second antigen binding site is selected among Fab fragments and VHH binding fragments; and
at least one of the first and the second antigen binding sites is a VHH fragment.

2. The compound according to claim 1, subject to one of the provisos:
a) One of the first and the second antigen binding site is a Fab fragment and the other antigen binding site is a VHH; or
b) The first and the second antigen binding sites are VHHs.

3. The compound according to claim 1 or 2, further comprising a tetramerization domain.

4. The compound according to any of claims 1 to 3, further comprising a third or subsequent binding site.

5. The compound of claim 4, wherein the third or subsequent binding site are tumor antigen binding sites.

6. The compound of claim 5, wherein the third or subsequent tumor binding sites are formed by a second or subsequent VHH fragment,

7. The compound of claim 5 or 6, wherein the third or subsequent binding site(s) are identical to the first binding site.

8. The compound according to any of claim 1-7, wherein the first binding site is capable of binding HER2, B7-H3, CA6, CD138, CD20, CD19, CD22, CD27L, CD30, CD33, CD37, CD38, CD47, CD56, CD66e, CD70, CD74, CD79b, BAFF, BAFFR, EGFR, EGFRvIII, FRα, GCC, GPNMB, Mesothelin, MUC16, NaPi2b, Nectin 4, PSMA, STEAP1, Trop-2, 5T4, AGS-16, alpha v beta6, CA19.9, CAIX, CD138, CD174, CD180, CD227, CD326, CD79a, CEACAM5, CRIPTO, DLL3, DS6, Endothelin B receptor, FAP, GD2, Mesothelin, PMEL 17, SLC44A4, TENB2, TIM-1, CD98, Endosialin/CD248/TEM1, Fibronectin Extra-domain B, LIV-1, Mucin 1, p-cadherin, peritosin, Fyn, SLTRK6, Tenascin c, VEGFR2, or PRLR.

9. The compound of any of the preceding claims, wherein the first antigen binding site is a Fab derived from one of 8H9 and 3F8.

10. The compound of any of claims 1-8, wherein the first antigen binding site is a VHH fragment.

11. The compound of claim 10, wherein the first antigen binding site comprises a sequence selected among amino acids 1-115 of SEQ ID NO: 10, 1-124 of SEQ ID NO: 12, 1-124 of SEQ ID NO: 16, 1-115 of SEQ ID NO: 19, 1-126 of SEQ ID NO: 20, 1-120 of SEQ ID NO: 21, 1-120 of SEQ ID NO: 22, 237-360 of SEQ ID NO: 23, 244-367 of SEQ ID NO: 25 or 1-117 of SEQ ID NO: 26.

12. The compound of any of the preceding claims, wherein the second antigen binding site is capable of binding DOTA, DOTAM, a DOTA derivative binding a metal ion or a DOTAM derivative binding a metal ion.

13. The compound of claim 12, wherein the second antigen binding site is a Fab derived from 2D12.5.

14. The compound of claims 12-13, wherein the second antigen binding site comprises amino acids 133-347 of sequence of SEQ ID NO 10 and the amino acids 1-222 of SEQ ID NO: 11.

15. The compound of claim 12, wherein the second antigen binding site comprises amino acids 146-364 of SEQ ID NO: 16 and 1-224 of SEQ ID NO: 17.

16. The compound of any of the preceding claims, wherein the tetramerization domain is selected among p53, p63, p73, hnRNPC, SNAP-23, StefinB, KCNQ4, CBFA2T1 domains and domains having at least 80% sequence identity to one of these domains.

17. The compound of any of the preceding claims, wherein the tetramerization domain is a domain with an amino acid sequence that differs from the sequence of amino acids 6-36 of SEQ ID NO: 1 by 1, 2, 3, 4, 5 or 6 substitutions selected among following substitutions:
E6V, Q, K, G, D or A;
Y7S, N, H, F, D or C;
F8Y, V, S, L, I or C;
T9S, P, N or A;
L10V, I or F;
Q11R, L, K, H or E;
I12V, T, M, L or F;
R13S, P, L, H, G or C;
G14W, R or A;
R15S, P, L, H, G or C;
E16V, Q, K, G, D or A;
F18Y, V, S, L, I or C;
E19V, Q, K, G, D or A;
M20V, T, R, L, K or I;
F21L or I;
R22L or G;
E23V, Q, K, G, D or A;
L24M;
N25S, I or D;
E26V, Q, K, G, D or A;
A27V, T, S, G or D;
L28W, V, M or F;
E29Q, G or D;
L30V, R, I, H or F;
K31T, R, Q, N, M or E;
D32Y, V, N, H, G or A;
A33V, T, S, P, G or D;
Q34R, L, K, H or E;
using the numbering of SEQ ID NO: 1.

18. The compound of claim 15 or 16, wherein the tetramerization domain comprises the sequence of amino acids 6-36 of SEQ ID NO 1.

19. The compound according to any of the preceding claims, wherein the first binding site is capable of binding HER2 and the second binding site is capable of binding DOTA.

20. The compound according to any of claims 1-18, wherein the first binding site is capable of binding CD38 and the second binding site is capable of binding DOTA.

21. The compound according to any of the claims 1-20, selected among compounds comprising the sequences of:
a. SEQ ID NO: 12 and SEQ ID NO: 11;
b. SEQ ID NO: 10 and SEQ ID NO: 11
c. SEQ ID NO: 12 and SEQ ID NO: 14;
d. SEQ ID NO: 12 and SEQ ID NO: 15;
e. SEQ ID NO: 16 and SEQ ID NO: 17;
f. SEQ ID NO: 16 and SEQ ID NO: 18;
g. SEQ ID NO: 19 and SEQ ID NO: 27;
h. SEQ ID NO: 20 and SEQ ID NO: 27;
i. SEQ ID NO: 21 and SEQ ID NO: 27;
j. SEQ ID NO: 22 and SEQ ID NO: 27;
k. SEQ ID NO: 26 and SEQ ID NO: 27
l. SEQ ID NO: 23 and SEQ ID NO: 27;
m. SEQ ID NO: 24 and SEQ ID NO: 25;
n. SEQ ID NO: 23 and SEQ ID NO: 25;
o. SEQ ID NO: 12 and SEQ ID NO: 13;
p. SEQ ID NO: 28 and SEQ ID NO: 29;
q. SEQ ID NO: 30 and SEQ ID NO: 31; and
r. SEQ ID NO: 30 and SEQ ID NO:32.

22. A composition comprising a compound of any of the preceding claims.

23. The composition of claim 22 being a pharmaceutical composition.

24. A method of treating and/or diagnosing cancer comprising the steps of:
i. Administering a compound according to claims 1-22 or a composition according to claim 23 or 24 to a person in need of treatment and/or diagnosis.
ii. Administering a radionuclide bound to the chelator recognized by the compound.

25. The method of claim 24, further comprising the step of:
iii. Scanning the patient to detect the location of radioactivity.

26. The method of claim 24 or 25, wherein the cancer is selected from osteosarcoma, neuroblastoma, liposarcoma, fibrosarcoma, carcinoma, malignant fibrous histiocytoma, leiomyosarcoma, spindle cell sarcoma, brain tumor, small cell lung cancer, retinoblastoma, HTLV-1 infected T cell leukemia, breast cancer, colon cancer, prostate cancer, T-cell and B-cell lymphomas, glioblastoma multiforme, malignant glioma, Head and Neck cancer, solid tumors and non-small-cell lung cancer.

27. The method of claim 24-25, wherein the cancer is breast cancer and the first binding site is capable of binding HER2.

28. The method of claim 27, wherein the compound is a compound according to claim 19.

29. The method of claim 24-26, wherein the cancer is a CD38 positive cancer, and the first binding site is capable of binding CD38.

30. The method of claim 29, wherein the compound is a compound according to claim 20.

31. The method of claims 24-30 further comprising a second and optional one or more subsequent administration(s) of radionuclide bound to a chelator.

32. A nucleic acid encoding a compound of claims 1-21.

33. An expression vector comprising the nucleic acid of claim 32.

34. A host cell comprising the nucleic acid of claim 33.

35. A method of producing the compound of claims 1-21, comprising the steps of
i. Providing a host cell of claim 34,
ii. Cultivating the host cell under conditions leading to expression of the nucleic acid encoding the compound, and
iii. Recovering the compound from the cultivation broth.

36. The method of claim 35, wherein the compound is a compound of claim 2 a), comprising the steps of
i. Providing a host cell comprising a first nucleic acid encoding a first polypeptide chain of the compound, and a second nucleic acid encoding the second polypeptide chain of the compound,
ii. Cultivating the host cell under conditions leading to expression of the two nucleic acids, and
iii. Recovering the compound from the cultivation broth.

37. The method of claim 35 or 36, wherein the host cell is HEK cell line.


SEQUENCE LISTING (32 sequences)

SEQ ID NO: 1 - protein, 39 aa, Homo sapiens
KPLDGEYFTLQIRGRERFEMFRELNEALELKDAQAGKEP

SEQ ID NO: 2 - protein, 57 aa, Homo sapiens
RSPDDELLYLPVRGRETYEMLLKIKESLELMQYLPQHTIETYRQQQQQQHQHLLQKQ

SEQ ID NO: 3 - protein, 52 aa, Homo sapiens
RHGDEDTYYLQVRGRENFEILMKLKESLELMELVPQPLVDSYRQQQQLLQRP

SEQ ID NO: 4 - protein, 27 aa, Homo sapiens
QAIKKELTQIKQKVDSLLENLEKIEKE

SEQ ID NO: 5 - protein, 54 aa, Homo sapiens
STRRILGLAIESQDAGIKTITMLDEQKEQLNRIEEGLDQINKDMRETEKTLTEL

SEQ ID NO: 6 - protein, 97 aa, Homo sapiens [sequence omitted]

SEQ ID NO: 7 - protein, 30 aa, Homo sapiens
DEISMMGRVVKVEKQVQSIEHKLDLLLGFY

SEQ ID NO: 8 - protein, 60 aa, Homo sapiens
TVAEAKRQAAEDALAVINQQEDSSESCWNCGRKASETCSGCNTARYCGSFCQHKDWEKHH

SEQ ID NO: 9 - protein, 5 aa, synthetic construct
GGGGS

SEQ ID NO: 10 - protein, 347 aa, synthetic construct
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSSGGGSGGGSGGGSGGGGSQAVVTQESALTTSPGETVTLTCRSSTGAVTTSNYANWVQEKPDHLFTGLIGGNNNRPPGVPARFSGSLIGDKAALTIAGTQTEDEAIYFCALWYSNHWVFGGGTRLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSPAECS

SEQ ID NO: 11 - protein, 284 aa, synthetic construct
HVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSKPLDGEYFTLQIRGRERFEMFRELNEALELKDAQAGKEPGGSGGAPHHHHHH

SEQ ID NO: 12 - protein, 360 aa, synthetic construct
DVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGIVAGRIPAEYADWGQGTQVTVSSGGGGSGGGGSGGGGSGGGGGSQAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 13 - protein, 230 aa, synthetic construct
HVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGHHHHHH

SEQ ID NO: 14 - protein, 419 aa, synthetic construct
DVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGIVAGRIPAEYADWGQGTQVTVSSGGGGSGGGGSGGGGSGGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSKPLDGEYFTLQIRGRERFEMFRELNEALELKDAQAGKEPGGS

SEQ ID NO: 15 - protein, 368 aa, synthetic construct
DVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGIVAGRIPAEYADWGQGTQVTVSSGGGGSGGGGSGGGGSGGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCG

SEQ ID NO: 16 - protein, 364 aa, synthetic construct
DVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGIVAGRIPAEYADWGQGTQVTVSSGGGGSGGGGSGGGGSGGGGGSDIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 276 aa, synthetic construct
VTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSKPLDGEYFTLQIRGRERFEMFRELNEALELKDAQAGKEPGGS

SEQ ID NO: 18 - protein, 225 aa, synthetic construct
VTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCG

SEQ ID NO: 19 - protein, 351 aa, synthetic construct
QVQLVDSGGGLVQAGGSLRLSCVASGALISRRGMGWYRQAPGRQRELVAGISSDGTTNYAQSVEGRFTISRDNAKRSVYLQMNSLKPEDTAVYYCHARTRWKSYWGQGTQVTVSSGGGGSGGGGSGGGGSGGGGGSQAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 20 - protein, 362 aa, synthetic construct
QVQLQESGGGLVQAGGSLRLSCAASGRTSSSSTMAWFRQAPGKEREFVAAITLSGGSTHYADSAKGRFTISRESAKNTVYLQMNSLKPEDTADYYCAARRWSNNRGGYDRAGYDYWGQGTQVTVSSGGGGSGGGGSGGGGSGGGGGSQAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 21 - protein, 356 aa, synthetic construct
EVQLVESGGGLVQPGGSLRLSCVASGFTFADYAMSWVRQAPGKGLQWVSSISYNGDTTYYAESMKDRFTISRDNAKNTLYLQMNSLKSEDTAVYYCASSGSYYPGHFESWGQGTQVTVSSGGGGSGGGGSGGGGSGGGGGSQAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 22 - protein, 356 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSGGGGSGGGGSGGGGSGGGGGSQAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 23 - protein, 360 aa, synthetic construct
QAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSGGGGSGGGGSGGGGSGGGGGSDVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGIVAGRIPAEYADWGQGTQVTVSS

SEQ ID NO: 24 - protein, 215 aa, synthetic construct
QAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 25 - protein, 419 aa, synthetic construct
HVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGGSDVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGIVAGRIPAEYADWGQGTQVTVSSGGGGSGGGGSKPLDGEYFTLQIRGRERFEMFRELNEALELKDAQAGKEPGGS

SEQ ID NO: 26 - protein, 352 aa, synthetic construct
DVQLVESGGGLVQPGGSLRLSCAASGFTFSRYWMGWFRQAPGKGVEWVSTINSGGGSTYYADSVKGRFTISRDNAKNTLYLQLNNLKTEDTAMYYCAKEQWRTGSRGQGTQVTVSSSGGGGSGGGGSGGGGSGGGGSQAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 27 - protein, 274 aa, synthetic construct
HVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSKPLDGEYFTLQIRGRERFEMFRELNEALELKDAQAGKEPGGS

SEQ ID NO: 28 - protein, 359 aa, synthetic construct
DVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGIVAGRIPAEYADWGQGTQVTVSSGGGGSGGGGSGGGGSGGGGSQAVVTQEPSLTVSPGGTVTLTCGSSTGAVTASNYANWVQQKPGQAPRGLIGGHNNRPPGVPARFSGSLLGGKAALTLLGAQPEDEAEYYCALWYSDHWVIGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 29 - protein, 278 aa, synthetic construct
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSKPLDGEYFTLQIRGRERFEMFRELNEALELKDAQAGKEPGGSGGAP

SEQ ID NO: 30 - protein, 347 aa, synthetic construct
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSSGGGSGGGSGGGSGGGGSQAVVTQEPSLTVSPGGTVTLTCGSSTGAVTASNYANWVQQKPGQAPRGLIGGHNNRPPGVPARFSGSLLGGKAALTLLGAQPEDEAEYYCALWYSDHWVIGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 31 - protein, 414 aa, synthetic construct
QVQLVESGGGLVQAGGSLRLSCAASGRTFSRQVMGWFRQAPGKERAFVAAISGSGGTTMYAGSVKGRFTISKDNAKNMVYLQMNSLKPEDTAVYYCNTVTPYDSWGQGTQVTVSSGGGGSGGGGSGGGGSGGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSKPLDGEYFTLQIRGRERFEMFRELNEALELKDAQAGKEPGGSGGAP

SEQ ID NO: 32 - protein, 424 aa, synthetic construct
QVQLVESGGGLVQSGDSLRLSCGWSGRTFSGYAMGWFRKAPGKDYEFVAAISVSETKTRYADSIRGRFTISRDNAKNTVYLQMNSLKPEDTAVYYCAASRSGVVGGREPTRYNYWGQGTQVTVSSGGGGSGGGGSGGGGSGGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSKPLDGEYFTLQIRGRERFEMFRELNEALELKDAQAGKEPGGSGGAP